(19) United States
(12) Patent Application Publication
(10) Pub. No.: US 2014/0287432 A1
(43) Pub. Date: Sep. 25, 2014

(54) Diagnosis, Prevention and Treatment of Disorders Characterized by Undesirable Cell Proliferation

(71) Applicant: Maria de Lourdes Higuchi, Sao Paulo (BR)

(72) Inventor: Maria de Lourdes Higuchi, Sao Paulo (BR)

(21) Appl. No.: 14/216,917

(22) Filed: Mar. 17, 2014

Related U.S. Application Data
(60) Provisional application No. 61/852,305, filed on Mar. 15, 2013.

(51) Int. Cl. C12Q 1/37; G01N 33/573

(52) U.S. Cl. CPC C12Q 1/37; G01N 33/573; G01N 2800/32; G01N 2800/325
USPC 435/7.4; 435/23

(57) ABSTRACT

The present invention relates to methods for the diagnosis, prevention and treatment of heart disease or heart failure in a subject. The present invention also relates to methods for the diagnosis, prevention and treatment of atherosclerosis with vulnerable plaque in a subject. Furthermore, the present invention relates to methods for the diagnosis, prevention and treatment of cardiomyopathies resulting from Chagas disease.

### PRIORITY CLAIM

This application claims priority to U.S. Provisional Application Ser. No. 61/852,305, filed Mar. 15, 2013, which is incorporated by reference in its entirety herein.

### 1. INTRODUCTION

The present invention relates to methods for the diagnosis, prevention and treatment of heart disease or heart failure in a subject. The present invention also relates to methods for the diagnosis, prevention and treatment of atherosclerosis with vulnerable plaque in a subject. Furthermore, the present invention relates to methods for the diagnosis, prevention and treatment of cardiomyopathies resulting from Chagas disease.

### 2. BACKGROUND

Current treatment for atherosclerosis involves lipid-lowering medications and drugs that affect lipid metabolism, including statins, bile acid absorption inhibitors, cholesterol absorption inhibitors, fibrates and antioxidants such as probucol, among others. (Zipes et al. Eds., 2005, Braunwald's Heart Disease, Elsevier Saunders, Philadelphia). These treatment regimens are based, at least in part, on the theory that oxidized lipoproteins are the main causative factor of atherosclerosis. However, the exact mechanism by which cholesterol oxidizes is still not fully understood.

Archaea are ancient microorganisms, but have been characterized only relatively recently. See, Woese et al., Proc Natl. Acad. Sci. U.S.A. 74: 5088-5090 (1977). They inhabit extreme environments and are constituted by lipid monolayer membranes. Rich alkaline atmosphere with sodium ions and metals prevents proliferation of other bacteria, but is favorable to arehaea's growth. Archaea have been isolated from alkaline waters from the Dead Sea, the Great Salt Lake and Yellowstone National Park. They have a small size, can-just barely-be viewed with an optical microscope, and observation of structural details requires electron microscopy. See, Howland et al., The surprising archaea. Discovering another domain of life, Oxford University Press (New York, 2000). Some are considered hyperthermophilic as they survive in very high temperatures.

Another unusual characteristic of some archaea is that they appear to use metal as an energy source. See, Amend et al., F.E.M.S. Microbiol. Rev. 25: 175-243 (2001). It is considered that archaea usually need an anaerobic or nearly anaerobic environments to carry out oxidation-reduction reactions with participation of different chemical compounds, including metals.

A new kind of extremely small archaea, which is dependent on bigger archaea, has also been described and named “nanoarchaea.” See, Huber J et al., Nature 417: 63-67 (2002). With the exception of archaea that reside in the mammalian intestine and produce methane gases, there is no report of archaea existing within plants or animals. See, Florin T H J et al., Am. J. Gastroenterol. 95: 2872-2879 (2000).

### 3. SUMMARY OF THE INVENTION

The present invention relates to compositions and methods for the reduction of atherosclerotic plaques. The present invention also relates to methods and compositions for diagnosing and treating subjects with vulnerable atherosclerotic plaques. Without being limited by theory, it is based on the hypothesis that the presence of mycoplasma and one or more other microorganism promotes atheroma formation. The present invention is also based on the discovery that Mycoplasma pneumoniae and oxidized LDL (low-density lipoprotein) antigens co-localize in microparticles in serum of subjects with vulnerable atherosclerotic plaques (and thus subjects at risk for acute myocardial infarction) at higher levels than in subjects with stable atherosclerotic plaques or subjects without atherosclerosis. The present invention is also based on the discovery that Chlamydia pneumoniae and archaea collagenase (e.g., Archaemetzincin-1, Archeobacterial metalloproteinase-like protein 1, AMZ1) antigens co-localize in microparticles in serum of subjects with vulnerable atherosclerotic plaques at higher levels than in subjects with stable atherosclerotic plaques or subjects without atherosclerosis.

The compositions and methods of the invention may also be used to decrease the level of one or more of total serum cholesterol, triglycerides, serum LDL cholesterol, and/or serum HDL cholesterol.

In one non-limiting embodiment of the invention, a composition comprises an agent that removes sialic acid residues, a metal chelator, and optionally one or more purified plant extracts.

In certain embodiments of the invention, a composition comprises a protein capable of removing sialic acid residues, such as a neuraminidase enzyme and/or a trans-sialidase enzyme; a metal chelator, preferably pyrrolidine dithiocarbamate (PDTC), along with one or more purified plant extracts. The purified plant extract may be derived from a plant selected from the group consisting of Allium sativum (garlic), Ginkgo biloba, tomato, orchids of the genus Cymbidium and Dendrobium, for example, Cymbidium ssp, Dendrobium nobik and Dendrobium moschatum; guava, ginseng, for example, Pfaffia panieulata (Brazilian ginseng); Zingiber officinale (ginger), and tobacco, wherein the purified extract comprises particles containing DNA or RNA, such as an archaea, nanoarchaea and/or archaeosome.

In certain embodiments, the methods of the present invention include methods of diagnosing vulnerable atherosclerotic plaque in a subject, wherein the method comprises detecting the co-localization of Mycoplasma pneumoniae and oxidized LDL in microparticles in a serum, plasma or blood sample from the subject. In certain embodiments, the method further comprises determining that the level of co-localized Mycoplasma pneumoniae and oxidized LDL is greater in the subject being tested than in a subject with stable atherosclerotic plaque or in subjects without atherosclerosis (or compared to a reference value determined using subjects with stable atherosclerotic plaque or without atherosclerosis).

In certain embodiments, the subject diagnosed with vulnerable atherosclerotic plaque is in treatment for malignant neoplasia.

In certain embodiments, the methods of the present invention include methods of diagnosing vulnerable atherosclerotic plaque in a subject, wherein the method comprises detecting the co-localization of Chlamydia pneumoniae and archaea collagenase (e.g., Archaemetzincin-1, Archeobacterial metalloproteinase-like protein 1, AMZ1) in microparticles in a serum, plasma or blood sample from the subject. In certain embodiments, the method further comprises determining that the level of co-localized Chlamydia pneumoniae and an archaea collagenase (e.g., Archaemetzincin-1, Archeobacterial metalloproteinase-like protein 1, AMZ1) is greater than in a subject with stable atherosclerotic plaque or in subjects without atherosclerosis (or compared to a reference value determined using subjects with stable atherosclerotic plaque or without atherosclerosis).

In certain embodiments, the subject diagnosed with vulnerable atherosclerotic plaque is in treatment for malignant neoplasia.

The present invention also provides methods for increasing the number of non-pathogenic archaea and/or nanoarchaea in a plant extract, while also decreasing the number of pathogenic archaea and/or nanoarchaea in the plant extract. In one embodiment, the non-pathogenic archaea and/or nanoarchaea in a plant extract are increased and the pathogenic archaea and/or nanoarchaea in the plant extract are decreased by aging the plant extract, and then diluting the plant extract with thermal water, followed by an additional aging period.

In certain embodiments, the non-pathogenic archaea and/or nanoarchaea comprise protein, for example, archaea collagenase (e.g., Archaemetzincin-1, Archeobacterial metalloproteinase-like protein 1, AMZ1).

The present invention also provides for methods for the diagnosis, prevention and treatment of disorders characterized by undesirable cell proliferation, for example, atherosclerosis and mycoplasma associated diseases. In one embodiment, such diagnosis includes detecting the presence of a microorganism or microbe in the serum, blood or plasma, or an atherosclerotic lesion of a subject. In other embodiments, such diagnosis includes detecting mycoplasma or mycoplasma lipoprotein in the serum, blood or plasma, or an atherosclerotic lesion of a subject. In other embodiments, diagnosis includes detecting chlamydia or chlamydia lipopolysaccharide (LPS) in the serum, blood or plasma, or an atherosclerotic lesion of a patient. In other embodiments, diagnosis includes detecting pathogenic archaea in the serum, blood or plasma, or an atherosclerotic lesion of a subject. In other embodiments, diagnosis includes detecting C reactive protein (CRP) in the serum, blood or plasma, or an atherosclerotic lesion of a patient.

In some embodiments, the methods of treating disorders characterized by undesirable cell proliferation, for example, atherosclerosis and mycoplasma associated diseases, according to the present invention comprise administering an amount of a composition according to the present invention to a patient in need of such treatment in an amount effective to reduce or inhibit one or more symptoms of the disorder characterized by undesirable cell proliferation.

The present invention also provides for methods for the diagnosis, prevention and treatment of cancer. In one embodiment, such diagnosis includes detecting the presence of a microorganism or microbe in the serum, blood or plasma, or neoplasia intima of a subject. In other embodiments, such diagnosis includes detecting mycoplasma or mycoplasma lipoprotein, in the serum, blood or plasma, or neoplasia intima of a subject. In other embodiments, diagnosis includes detecting chlamydia or chlamydia lipopolysaccharide (LPS) in the serum, blood or plasma, or neoplasia intima of a subject. In other embodiments, diagnosis includes detecting pathogenic archaea in the serum, blood or plasma, or neoplasia intima of a subject. In other embodiments, diagnosis includes detecting the presence of mycoplasma or mycoplasma lipoprotein, chlamydia or chlamydia lipopolysaccharide, or pathogenic archaea in a cell culture of a subject sample.

In some embodiments, the methods of treating cancer according to the present invention comprise administering an amount of a composition according to the present invention to a patient in need of such treatment in an amount effective to reduce or inhibit the presence of cancer or tumor cells.

The present invention also provides for methods for the diagnosis, prevention and treatment of cardiotoxicity, heart disease or heart failure in cancer patients undergoing treatment for cancer. In one embodiment, such diagnosis includes detecting the presence of a microorganism or microbe in the serum, blood or plasma of a patient. In other embodiments, such diagnosis includes detecting mycoplasma or mycoplasma lipoprotein in the serum, blood or plasma of a patient. In other embodiments, diagnosis includes detecting chlamydia or chlamydia lipopolysaccharide (LPS) in the serum, blood or plasma of a patient. In other embodiments, diagnosis includes detecting pathogenic archaea in the serum, blood or plasma of a patient.

In some embodiments, the methods of treating cardiotoxicity, heart disease or heart failure in cancer patients undergoing treatment for cancer, according to the present invention, comprise administering an amount of a composition according to the present invention to a patient in need of such treatment in an amount effective to reduce or inhibit one or more symptoms of cardiotoxicity, heart disease or heart failure.

The present invention provides for methods for the diagnosis, prevention and treatment of heart disease, for example, cardiomyopathies resulting from Chagas disease, for example, dilated cardiomyopathy or chronic cardiopathy. In one embodiment, diagnosis of chagasic cardiomyopathies includes the detection of a microorganism or microbe in the serum, blood or plasma of a subject. In other embodiments, diagnosis of chagasic cardiomyopathies includes the detection of mycoplasma or mycoplasma lipoprotein, chlamydia or chlamydia lipopolysaccharide in the serum, blood or plasma of a subject. In further embodiments, the microorganism or microbe is associated with pathogenic archaea, for example, empty pathogenic archaea, archaea with electron dente content or archaea with electron lucent content.

In some embodiments, the methods of treating cardiomyopathies resulting from Chagas disease, according to the present invention comprise administering an amount of a composition according to the present invention to a patient in need of such treatment in an amount effective to reduce or inhibit one or more symptoms of a cardiomyopathy resulting from Chagas disease.

In other embodiments, diagnosis includes detecting archaeal-like organelles such as archaeosomes or archaeal proteasomes, and/or archaeal nucleic acid in a sample from a patient, for example, an endomyocardial biopsy (EB), serum, blood or plasma sample. In one embodiment, diagnosis includes detecting electron dense lipidic organelles in the sample. In other embodiments, the diagnosis further includes detecting archaeal nucleic acid in the sample. In further embodiments, the microbes are associated with pathogenic archaea, for example, empty pathogenic archaea, or archaea with electron lucent content.

In certain embodiments, detecting a greater number of microparticles (e.g., electron dense microparticles as described herein) which comprise protein, for example, collagenase, such as archaea collagenase (e.g., Archaemetzincin-1, Archeobacterial metalloproteinase-like protein 1, AMZ1) in a sample from a first subject compared to a sample from a second subject with heart failure associated with Chagas disease, indicates a diagnosis of the indeterminate form of Chagas disease in the first subject.

In certain embodiments, detecting a greater amount or level of protein, for example, collagenase, such as archaea collagenase (e.g., Archaemetzincin-1, Archeobacterial metalloproteinase-like protein 1, AMZ1) external to microparticles (e.g., electron dense microparticles as described herein) in a sample from a first subject compared to a sample from a second subject with the indeterminate form of Chagas disease, indicates a diagnosis of heart failure associated with Chagas disease in the first subject.

In certain embodiments, the present invention provides a method for diagnosing heart disease or heart failure in a first subject comprising (a) obtaining a first sample from the first subject; (b) obtaining a second sample from a second subject, wherein the second subject does not have heart disease or heart failure; and (c) detecting the presence and/or determining the number of microparticles in the first and second samples, wherein the presence of a fewer number of electron dense microparticles comprising archael collagenase in the first sample compared to the second sample indicates a diagnosis of heart disease or heart failure.

In certain embodiments, the second sample from the second subject comprises microparticles, for example, electron dense and/or electron lucent microparticles.

In certain embodiments, the serum, blood, plasma and/or endomyocardial tissue of the second subject comprises microparticles, for example, electron dense and/or electron lucent microparticles.

In certain embodiments, the present invention provides a method for diagnosing heart disease or heart failure in a subject comprising (a) obtaining a sample from the subject; (b) detecting the presence of microparticles in the sample; and (c) comparing the number of electron dense microparticles comprising archael collagenase in the sample with a reference value determined by measuring the number of electron dense microparticles comprising archael collagenase in one or more control subject(s) that does (do) not suffer from heart disease or heart failure, wherein the presence of a fewer number of electron dense microparticles comprising archael collagenase in the first sample compared to the second sample indicates a diagnosis of heart disease or heart failure.

In certain embodiments, the control subject(s) have the indeterminate form of Chagas disease.

In some embodiments of the present invention, diagnosis comprises detecting the presence of mycoplasma or mycoplasma lipoprotein, chlamydia or chlamydia lipopolysaccharide, pathogenic archaea nucleic acid, or pathogenic archaea antigen in a serum, blood or plasma sample from a subject.

The present invention also provides for in vitro methods for selecting a composition of the present invention for use in treating a disorder characterized by undesirable cell proliferation, heart disease or heart failure caused by injury or Chagas disease, dilated cardiomyopathy, cancer, cardiotoxicity, and heart disease or heart failure during cancer treatment. In one embodiment, the in vitro method comprises assaying the effect of a composition of the present invention in reducing the presence of a microorganism or microbe in a sample from a subject, for example, a serum, blood or plasma sample, or a cell culture of a subject sample, for example, a cancer cell sample. In one embodiment, the composition that is most effective in reducing the presence of microorganisms or microbes in the sample is selected for use in treating a subject in need of such treatment.

### 4. BRIEF DESCRIPTION OF THE FIGURES

FIG. 1A-J. (A-E) shows macroscopic aortic atheroma plaques (arrows) and (F-J) shows Chlamydia pneumoniae positive antigen expression in aortal intimal areas (arrows) of rabbits fed a 1% cholesterol diet and submitted to different anti-atherosclerotic treatments. Group II (A, F) received no treatment, Group III (B, G) was treated with trans-sialidase (“TS”)+pyrrolidine dithiocarbamate (“PDTC”), Group IV (C, H) was treated with TS+PDTC+Allium sativum (“AS”), Group V (D, I) was treated with TS+PDTC+AS+Ginkgo biloba (“GB”), and Group VI (E, J) was treated with TS+PDTC+AS+GB+Zingiber officinale (“ZO”).

FIG. 2 shows the nucleotide sequence of a plasmid encoding the catalytic trans-sialidase unit of trans-sialidase from Trypanosoma cruzi (SEQ ID NO:3). The letters in capital represent the pET14b plasmid and the underlined letters correspond to the position of the oligonucleotides used to amplify the Trypanosoma cruzi clone.

FIG. 3 shows the amino acid sequence of the protein encoded by the nucleic acid sequence depicted in FIG. 2. (SEQ ID NO:4). In bold are the amino acids not found in the original clone.

FIG. 4 shows small dark electron-dense nanoarchaea of between 0.03-0.15 μm in diameter.

FIG. 5 shows dark medium sized electron-dense archaea of between 0.5-1.1 μm in diameter, and large clear, empty archaea of between 1.0-2.4 μm in diameter.

FIG. 6 shows clear, empty archaea of between 0.15-2.0 μm in diameter.

FIG. 7 shows an electron micrograph of a human aortic aneurysm. The aortic aneurysm exhibits many round lipidic bodies in both the cytoplasm of macrophages and in the extracellular matrix. The round lipidic bodies are surrounded by immunogenic lymphocytes.

FIG. 8 shows a high magnification view of the round lipidic body described in FIG. 7. The round lipidic body exhibits a clear external membrane corresponding to the morphology of the large lipidic archaea microbes shown in FIGS. 5 and 6 (and also isolated from tobacco).

FIG. 9 shows an electron micrograph of the serum of a patient with symptoms of Lyme disease, but negative for Borrelia burgdorferi, showing two mycoplasmas that have one envoltory membrane and granulous electron dense material inside. On the top the mycoplasma in intimal contact with a structure with morphology of archaea characterized by thin delicate envoltory lipidic monolayer membrane and having an internal empty space, which is characteristic of the archaea present in tissue lesions.

FIG. 10A-B shows the presence of different primitive microbial bodies associated with empty pathogenic archaea in the serum of patients with dilated chagastic cardiomyopathy.

FIG. 11 shows that lysing neoplastic cells in vitro results in the release of intracellular infectious agents that are morphologically characteristic of mycoplasmas and archaeas.

FIG. 12A-D shows that the addition of a composition comprising nanoparticles to a cancer cell culture results in apoptosis of the neoplastic cells (A) and release of microbes into the extracellular medium from the apoptotic neoplastic cells (B). Addition of a composition comprising nanoparticles, trans-sialidase and PDTC increased the amount of neoplastic cell apoptosis (C), and reduced the amount of microbes released into the extracellular medium from the apoptotic neoplastic cells (D).

FIG. 13A-D shows the presence of archaea and other microbes present in a serum sample from a Chagas disease patient (A) and (13). The addition of a composition comprising nanoparticles from Ginkgo biloba, Zingiber officinalis, Golden root and orchid (Dendrobium moschatum), to the serum sample reduced the amount of archaea and other microbes present in the sample (C), and that the addition of 3 ul of trans-sialidase diluted 1,000,000 fold in association with PDTC to the nanoparticles further reduced the amount of archaea and other microbes present in the sample (D). The presence of archaea were determined using fluorescent Qdots.

FIG. 14 shows nanovesicles containing archaea DNA resembling archaeosomes that are present in ED MPs exhibiting Chlamydophila pneumonia antigens in association with oxidized LDL.

FIG. 15A-F shows co-localization of Mycoplasma pneumoniae and oxidized LDL antigens in microparticles in serum samples from subjects with (A) acute myocardial infarction, (C) stable atherosclerotic plaque, and (E) subjects that do not have atherosclerosis. Mycoplasma pneumoniae antigens were detected using an anti-Mycoplasma pneumoniae antibody and rhodamine staining, and oxidized LDL antigens were detected using anti-oxidized LDL antibody and fluorescein staining. FIGS. 15 B, D and F shows co-localization of Chlamydia pneumoniae and archaea collagenase (AMZ1) antigens in microparticles in serum samples from subjects with (B) acute myocardial infarction, (D) stable atherosclerotic plaque, and (F) subjects that do not have atherosclerosis. Archaea collagenase (AMZ1) antigens were detected using an anti-AMZ1 antibody and rhodamine staining, and Chlamydia pneumoniae antigens were detected using anti-Chlamydia pneumoniae antibody and fluorescein staining.

### 5. DETAILED DESCRIPTION OF THE INVENTION

For purposes of clarity, and not by way of limitation, the detailed description of the invention is divided into the following subsections:

(i) Methods of Diagnosis;

(ii) Therapeutic compositions;

(iii) Therapeutic uses; and

(iv) in vitro assay.

### 5.1 Methods of Diagnosis

### 5.1.1 Diagnosis of Diseases Associated with Undesirable Cell Proliferation and Fibrosis

The present invention provides for methods of diagnosing, preventing and treating a disorder characterized by undesirable cell proliferation and fibrosis, for example, atherosclerosis and mycoplasma associated diseases. In one embodiment of the invention, diagnosis of atherosclerosis and/or a mycoplasma associated disease is performed by detecting the presence of an agent in the serum, blood or plasma of a patient, wherein detection of the agent indicates the existence or likelihood of developing atherosclerosis and/or a mycoplasma associated disease. In other embodiments of the invention, diagnosis is performed by detecting the presence of the agent in an atherosclerotic lesion.

The presence of the agent may be detected, for example, using electron microscopy, fluorescence microscopy, immunohistochemistry, polymerase chain reaction (PCR) or any other method known in the art.

In some non-limiting embodiments, the agent is a microbe or a microbe antigen. In other embodiments, the agent is a nucleic acid, for example, a nucleic acid from a microbe.

In one non-limiting embodiment, the agent is mycoplasma or a mycoplasma lipoprotein, for example, Mycoplasma pneumoniae lipoprotein. In certain embodiments, the agent is Mycoplasma pulmonis lipoprotein.

In other non-limiting embodiments, the agent is chlamydia or a chlamydia lipopolysaccharide (LPS), for example, Chlamydia pneumoniae or Chlamydia pneumoniae LPS.

In other non-limiting embodiments, the agent is a pathogenic archaea.

In other non-limiting embodiments, the agent is C reactive protein (CRP).

In other non-limiting embodiments, the agent is spirochete.

In one non-limiting embodiment of the present invention, detecting the presence of mycoplasma, mycoplasma lipoprotein, CRP, or a combination thereof, in serum of a patient indicates a diagnosis of atherosclerosis, coronary artery disease (CAD), or the presence of a stable atheroma in the patient.

In other non-limiting embodiments of the present invention, detecting the presence of chlamydia, chlamydia LPS, pathogenic archaea, or a combination thereof, in serum of a patient indicates a diagnosis of acute myocardial infarction (AMI), or the presence of an unstable or ruptured atheroma in the patient.

In one non-limiting embodiment of the invention, the agent can be concentrated from the serum and plasma of a subject (e.g., a human patient) before detecting the presence of the agent. For example, serum can be separated from blood by incubating a blood sample in a water bath to form a blood clot. The serum can be isolated from the blood clot by centrifuging the sample to obtain a supernatant comprising the serum. The serum can then be prepared for electron microscopy analysis by fixing the serum (e.g., by combining 1 ml of serum with 1.0 ml of glutaraldehyde fixative and 1.0 ml of Osmium tetroxide), centrifuging the treated serum, and sectioning the resulting pellet. The supernatant can also be analyzed using electron microscopy for agent detection.

In certain non-limiting embodiments, the present invention provides for methods of diagnosing heart disease or heart failure in a first subject, wherein the method comprises detecting electron lucent microparticles in a sample from the subject, wherein the concentration or number of electron lucent microparticles detected in the sample is greater than the concentration or number of electron lucent microparticles detected in a sample from a second subject that does not have heart disease or heart failure, or a representative reference value obtained from one or more subject that does not have heart disease or heart failure.

In certain non-limiting embodiments, the electron lucent microparticles are electron lucent lipidic particles.

In certain non-limiting embodiments, the first subject has Chagas disease.

In certain non-limiting embodiments, the second subject has Chagas disease.

In certain non-limiting embodiments, the second subject has the indeterminate form of Chagas disease.

In certain non-limiting embodiments, the electron lucent microparticles have a diameter that is greater than about 100 nm, greater than about 125 nm, greater than about 150 nm, greater than about 175 nm, greater than about 200 nm, greater than about 250 nm, greater than about 500 nm, or greater than about 1 um. In certain non-limiting embodiments, the electron lucent microparticles have a diameter that is between about 100 nm and about 2 um, or between about 125 nm and about 1.5 um, or between about 150 nm and about 1 um, or between about 200 nm and about 500 nm.

In certain non-limiting embodiments, the electron lucent microparticles are associated with collagenase. In certain embodiments the collagenase is archaeal collagenase. In certain embodiments, the archaeal collagenase is Archaemetzincin-1, Archeobacterial metalloproteinase-like protein 1, AMZ1. In certain non-limiting embodiments, the concentration or number of electron lucent microparticles associated with collagenase in the sample from the first subject is greater than the concentration or number of electron lucent microparticles associated with collagenase in the sample from the second subject.

In certain non-limiting embodiments, the electron lucent microparticles comprise exosomes.

In certain non-limiting embodiments, the first and second subject samples are independently selected from the group consisting of serum, blood, plasma and endomyocardial biopsy.

In certain non-limiting embodiments, the present invention provides for a method of diagnosing heart disease or heart failure in a subject comprising detecting the presence of an agent in a sample from the subject, wherein the agent is selected from the group consisting of pathogenic archaea, pathogenic archaea nucleic acid, mycoplasma, chlamydia, C reactive protein, spirochete, virus, electron lucent microparticle, and combinations thereof, wherein presence of the agent indicates diagnosis of heart disease or heart failure.

In certain non-limiting embodiments, the electron lucent microparticle is an electron lucent lipidic particle.

In certain non-limiting embodiments, the method of diagnosing heart disease or heart failure further comprises obtaining a first sample from the subject; obtaining a second sample from a second subject who does not have heart disease or heart failure; and detecting the presence of electron lucent microparticles in the first and second samples, wherein the presence of a greater number of electron lucent microparticles in the first subject sample compared to the second subject sample indicates a diagnosis of heart disease or heart failure in the first subject.

In certain embodiments, the present invention provides a method for diagnosing heart disease or heart failure in a first subject comprising (a) obtaining a sample from the first subject; (b) detecting the presence and/or determining the number of microparticles in the sample; and (c) comparing the number of electron dense microparticles comprising archael collagenase in the sample with a reference value determined by measuring the number of electron dense microparticles comprising archael collagenase in one or more control subject(s) that does (do) not suffer from heart disease or heart failure, wherein the presence of a fewer number of electron dense microparticles comprising archael collagenase in the first sample compared to the second sample indicates a diagnosis of heart disease or heart failure.

In certain non-limiting embodiments, the heart disease or heart failure is associated with Chagas disease.

In certain non-limiting embodiments, the heart disease or heart failure is associated with cardiotoxicity associated with treatment of cancer.

In certain non-limiting embodiments, the subject has been diagnosed with dilated cardiomyopathy or lymphocytic myocarditis.

In certain non-limiting embodiments, each sample is independently selected from the group consisting of serum, blood, plasma and an endomyocardial sample.

In certain non-limiting embodiments, the electron lucent microparticles are associated with nucleic acid.

In certain non-limiting embodiments, the nucleic acid associated with the electron lucent microparticles is archaea nucleic acid.

In certain embodiments, the methods of the present invention include methods of diagnosing vulnerable atherosclerotic plaque in a subject, wherein the method comprises detecting the co-localization of Mycoplasma pneumoniae and oxidized LDL in microparticles in a serum, plasma or blood sample from the subject. In certain embodiments, the level of co-localized Mycoplasma pneumoniae and oxidized LDL is greater than in a subject with stable atherosclerotic plaque, or in subjects without atherosclerosis, or compared to a reference value determined using subjects with stable atherosclerotic plaque or without atherosclerosis.

In certain embodiments, the methods of the present invention include methods of diagnosing vulnerable atherosclerotic plaque in a subject, wherein the method comprises detecting the co-localization of Chlamydia pneumoniae and archaea collagenase (for example, Archaemetzincin-1, Archeobacterial metalloproteinase-like protein 1, AMZ1) in microparticles in a serum, plasma or blood sample from the subject. In certain embodiments, the level of co-localized Chlamydia pneumoniae and archaea collagenase (or example, Archaemetzincin-1, Archeobacterial metalloproteinase-like protein 1, AMZ1) is greater than in a subject with stable atherosclerotic plaque, or in subjects without atherosclerosis, or compared to a reference value determined using subjects with stable atherosclerotic plaque or without atherosclerosis.

In certain embodiments, the Mycoplasma pneumoniae, oxidized LDL, Chlamydia pneumoniae and/or archaea collagenase are detected using immunofluorescence, for example, but not limited to, ELISA, wherein a detectable antibody specific for the target allows for identifying the presence of the Mycoplasma pneumoniae, oxidized LDL, Chlamydia pneumoniae and/or archaea collagenase.

In certain embodiments, the level of Mycoplasma pneumoniae, oxidized LDL, Chlamydia pneumoniae and/or archaea collagenase detected in a sample can be determined using any method of quantification known in the art, for example, flow cytometry, proximity ligation assays (in situ PLA) such as Duolink®, and Proximity Extension Assay technology (PEA) assays such as Proseek®.

In certain embodiments, the subject diagnoses with vulnerable atherosclerotic plaque is at a greater risk of acute myocardial infarction than a subject not diagnosed with vulnerable atherosclerotic plaque, for example, a subject with stable atherosclerotic plaque.

In certain non-limiting embodiments, the method of diagnosing vulnerable atherosclerotic plaque comprises obtaining a first sample from a subject; obtaining a second sample from a second subject who does not have vulnerable atherosclerotic plaque; and detecting the presence of co-localized Mycoplasma pneumoniae and oxidized LDL in the first and second samples, wherein the presence of a greater number of co-localized Mycoplasma pneumoniae and oxidized LDL in the first subject sample compared to the second subject sample indicates a diagnosis of vulnerable atherosclerotic plaque in the first subject. As an alternative to obtaining a second sample a reference value determined using one or more subjects who do not have vulnerable atherosclerotic plaque may also be used.

In certain non-limiting embodiments, the method of diagnosing vulnerable atherosclerotic plaque comprises obtaining a first sample from a subject; obtaining a second sample from a second subject who does not have vulnerable atherosclerotic plaque; and detecting the presence of co-localized Chlamydia pneumoniae and archaea collagenase in the first and second samples, wherein the presence of a greater number of co-localized Chlamydia pneumoniae and archaea collagenase in the first subject sample compared to the second subject sample indicates a diagnosis of vulnerable atherosclerotic plaque in the first subject.

In certain embodiments, detecting a fewer number of microparticles (e.g., electron dense microparticles as described herein) which comprise protein, for example, collagenase, such as, but not limited to, archaea collagenase (e.g., Archaemetzincin-1, Archeobacterial metalloproteinase-like protein 1, AMZ1) in a sample from a first subject compared to a sample from a second subject without heart disease or heart failure, indicates a diagnosis of heart disease or heart failure in the first subject. In certain embodiments, the second sample from the second subject comprises microparticles, for example, electron dense and/or electron lucent microparticles. In certain embodiments, the serum, blood, plasma and/or endomyocardial tissue of the second subject comprises microparticles, for example, electron dense and/or electron lucent microparticles.

In certain embodiments, detecting a greater amount or level of protein, for example, collagenase, such as, but not limited to, archaea collagenase (e.g., Archaemetzincin-1, Archeobacterial metalloproteinase-like protein 1, AMZ1) external to microparticles (e.g., electron dense microparticles as described herein) in a sample from a first subject compared to a sample from a second subject without heart disease or heart failure (or a predetermined reference value), indicates a diagnosis of heart disease or heart failure associated in the first subject.

In certain embodiments, the present invention provides methods for diagnosing heart disease or heart failure in a first subject comprising (a) obtaining a first sample from the first subject (b) obtaining a second sample from a second subject, wherein the second subject does not have heart disease or heart failure; and (c) detecting the presence of microparticles in the first and second samples, wherein the presence of a fewer number of electron dense microparticles comprising archael collagenase in the first sample compared to the second sample indicates a diagnosis of heart disease or heart failure. In certain embodiments, the second sample from the second subject comprises microparticles, for example, electron dense and/or electron lucent microparticles. In certain embodiments, the serum, blood, plasma and/or endomyocardial tissue of the second subject comprises microparticles, for example, electron dense and/or electron lucent microparticles. As an alternative to obtaining a second sample a reference value determined using one or more subjects who do not have heart disease or heart failure may also be used.

### 5.1.2 Diagnosis of Cancer or Cardiotoxicity in Cancer Patients Undergoing Treatment for Cancer

The present invention also provides for compositions and methods for the diagnosis, prevention and treatment of cancer and cardiotoxicity, heart disease or heart failure in cancer patients undergoing treatment for cancer. In one embodiment of the invention, diagnosis of cancer or cardiotoxicity, heart disease or heart failure in cancer patients undergoing treatment for cancer is performed by detecting the presence of an agent in the serum, blood or plasma of a patient, wherein detection of the agent indicates the existence or likelihood of developing cancer or cardiotoxicity, heart disease or heart failure. In other embodiments of the invention, diagnosis is performed by detecting the presence of the agent in a neoplasia intima of a patient.

In other embodiments, diagnosis includes detecting the presence of the agent in a cell culture of a patient sample, for example a cell culture of a cancer cell sample from the patient.

The presence of the agent may be detected, for example, using electron microscopy, fluorescence microscopy, immunohistochemistry, polymerase chain reaction (PCR) or any other method known in the art.

In some non-limiting embodiments, the agent is a microbe or a microbe antigen. In other embodiments, the agent is a nucleic acid, for example, a nucleic acid from a microbe.

In one non-limiting embodiment, the agent is mycoplasma or a mycoplasma lipoprotein, for example, Mycoplasma pneumoniae or Mycoplasma pneumoniae lipoprotein. In certain embodiments, the agent is Mycoplasma pulmonis lipoprotein.

In other non-limiting embodiments, the agent is chlamydia or a chlamydia lipopolysaccharide (LPS), for example, Chlamydia pneumoniae or Chlamydia pneumoniae LPS.

In other non-limiting embodiments, the agent is a pathogenic archaea.

In other non-limiting embodiments, the agent is spirochete.

In one non-limiting embodiment of the present invention, detecting the presence of mycoplasma, mycoplasma lipoprotein or a combination thereof, in serum of a patient or in a neointima sample from a patient indicates a diagnosis of cancer or cardiotoxicity, heart disease or heart failure.

In one non-limiting embodiment of the invention, the agent can be concentrated from the serum and plasma of a subject (e.g., a human patient) before detecting the presence of the agent. For example, serum can be separated from blood by incubating a blood sample in a water bath to form a blood clot. The serum can be isolated from the blood clot by centrifuging the sample to obtain a supernatant comprising the serum. The serum can then be prepared for electron microscopy analysis by fixing the serum (e.g., by combining 1 ml of serum with 1.0 ml of glutaraldehyde fixative and 1.0 ml of Osmium tetroxide), centrifuging the treated serum, and sectioning the resulting pellet. The supernatant can also be analyzed using electron microscopy for agent detection.

In certain non-limiting embodiments, the present invention provides for a method for diagnosing heart disease or heart failure in a subject being treated for cancer comprising obtaining a first sample from the subject; obtaining a second sample from a second subject, wherein the second subject does not have heart disease or heart failure; detecting the presence of an agent in the first and second samples, wherein the agent is selected from the group consisting of pathogenic archaea, pathogenic archaea nucleic acid, mycoplasma, chlamydia, C. reactive protein, spirochete, virus, electron lucent microparticles, and combinations thereof, wherein a higher concentration of agent in the first sample compared to the second sample indicates a diagnosis of heart disease. As an alternative to obtaining a second sample a reference value determined using one or more subjects who do not have heart disease or heart failure may also be used.

In certain non-limiting embodiments of the present invention, the microparticles comprise a double surrounding membrane.

In certain non-limiting embodiments of the present invention, the microparticles comprise nucleic acid.

In certain non-limiting embodiments of the present invention, the microparticles comprise archaea nucleic acid.

### 5.1.3 Diagnosis of Chagasic Cardiomyopathy

Chagas disease is a tropical parasitic disease caused by the flagellate protozoan Trypanosoma cruzi. While most Chagas disease patients remain asymptomatic during their lifetimes, about 30% of infected patients develop dilated cardiomyopathy related to an apparently autoimmune myocarditis.

Archaea-like bodies have been found in association with Chagas disease, in two morphological types: one with electron dense lipidic content (EDL) and other with electron lucent content (ELC) (Higuchi et al. Mem Inst Oswaldo Cruz 2009; 104 (Suppl 1): 199-207.) Additionally, trypanosoma have been discovered to carry proteasomes from archaea, which has been hypothesized to have occurred through an evolutionary endosymbiotic mechanism. Proteasomes are organdies that degrade unneeded or damaged proteins by proteolysis.

The present invention provides for methods for the diagnosis, prevention and treatment of heart disease, for example, cardiomyopathies resulting from Chagas disease. In one embodiment, such diagnosis includes detecting the presence of an agent in the serum, blood or plasma of a patient with Chagas disease, wherein detection of the agent indicates the existence or likelihood of developing heart disease, for example, cardiomyopathies resulting from Chagas disease.

The presence of the agent may be detected, for example, using electron microscopy, fluorescence microscopy, immunohistochemistry, polymerase chain reaction (PCR) or any other method known in the art.

In some non-limiting embodiments, the agent is a microbe or a microbe antigen. In other embodiments, the agent is a nucleic acid, for example, a nucleic acid from a microbe.

In one non-limiting embodiment, the agent is mycoplasma or a mycoplasma lipoprotein, for example, Mycoplasma pneumoniae or Mycoplasma pneumoniae lipoprotein. In certain embodiments, the agent is Mycoplasma pulmonis lipoprotein.

In other non-limiting embodiments, the agent is chlamydia or a chlamydia lipopolysaccharide (LPS), for example, Chlamydia pneumoniae or Chlamydia pneumoniae LPS.

In other non-limiting embodiments, the agent is a pathogenic archaea.

In other non-limiting embodiments, the agent is spirochete.

In some embodiments, the agent is an archaeal-like organelles such as archaeosomes or archaeal proteasomes and/or archaeal nucleic acid in a sample from a patient.

In one embodiment, diagnosis includes detecting electron dense lipidic (EDL) archaeal organelles in a sample from a patient. In other embodiments, the diagnosis further includes detecting archaeal nucleic acid in the sample. In certain embodiments, the archaeal nucleic acid is present within the EDL organelle.

In other embodiments, diagnosis includes detecting electron lucent content (ELC) archaeal organelles in a sample from a patient. In other embodiments, the diagnosis further includes detecting archaeal nucleic acid in the sample. In certain embodiments, the archaeal nucleic acid is present within the ELC organelle.

In one embodiment, the sample is serum, blood or plasma. In other embodiments, the sample is an endomyocardial biopsy (EB).

In one non-limiting embodiment of the invention, the agent can be concentrated from the serum and plasma of a subject (e.g., a human patient) before detecting the presence of the agent. For example, serum can be separated from blood by incubating a blood sample in a water bath to form a blood clot. The serum can be isolated from the blood clot by centrifuging the sample to obtain a supernatant comprising the serum. The serum can then be prepared for electron microscopy analysis by fixing the serum (e.g., by combining 1 ml of serum with 1.0 ml of glutaraldehyde fixative and 1.0 ml of Osmium tetroxide), centrifuging the treated serum, and sectioning the resulting pellet. The supernatant can also be analyzed using electron microscopy for agent detection.

In one embodiment, detecting a negative correlation between the number of electron dense organelles versus the amount of archaeal nucleic acid outside of the electron dense organelles (for example, in the extracellular matrix) in a sample is indicative of a diagnosis of indeterminate asymptomatic form (IF) of Chagas disease.

In one embodiment, detecting a positive correlation between the number of electron dense organelles versus the amount of archaeal nucleic acid outside of the electron dense organelles (for example, in the extracellular matrix) in a sample is indicative of a diagnosis of Chagas disease related heart disease.

The present invention also provides for methods of diagnosing chagasic cardiomyopathies in a patient, for example, dilated cardiomyopathy or chronic cardiopathy. In one embodiment, diagnosing includes comparing a sample from a first patient with Chagas disease with a sample from a second patient with Chagas disease who does not have a chagasic cardiomyopathy, wherein the sample from the first patient comprises smaller archaeal organelles, for example electron dense archael organelles, than the sample from the second patient. In other embodiments, the sample from the first patient comprises less archaeal nucleic acid present outside of the archaeal organelles than the sample from the second patient. In other embodiments, the sample from the first patient comprises smaller archaeal organelles and less archaeal nucleic acid outside of the archaeal organelles than the sample from the second patient. As an alternative to obtaining a second sample a reference value determined using one or more subjects who do not have chagasic cardiomyopathy may also be used.

In one embodiment, the second patient has indeterminate asymptomatic form (IF) of Chagas disease.

In one embodiment, the samples from the first patient and second patient are endomyocardial biopsy samples.

In other embodiments, the samples from the first patient and second patient are serum, blood or plasma samples.

In other embodiments, diagnosis of chagasic cardiomyopathies includes the detection of microbes in the serum, blood or plasma of a patient. In further embodiments, the microbes are associated with empty pathogenic archaea.

In one embodiment, the empty pathogenic archaea comprise organelles that are electron lucent content bodies (ELC).

In other embodiments of the present invention, detection of pathogenic archaea, archaea nucleic acid, mycoplasm, spirochete and/or Chlamydia in the serum and/or myocardium of a heart transplant donor indicates a greater risk of mortality in the heart transplant recipient of the donor heart than a recipient who receives a heart from a donor with less pathogenic archaea, archaea nucleic acid, mycoplasm, spirochete and/or Chlamydia in the donor's serum and/or myocardium.

In other embodiments, the presence of lymphocytic infiltrate in the myocardium of a donor heart indicates a higher risk of mortality in the recipient of the donor heart than a recipient who receives a heart from a donor with less lymphocytic infiltrate in the myocardium of the donor heart.

In certain non-limiting embodiments, the present invention provides for methods of diagnosing the indeterminate form of Chagas disease in a first subject, wherein the method comprises detecting electron dense microparticles in a sample from the subject, wherein the concentration or number of electron dense microparticles detected in the sample is greater than the concentration or number of electron dense microparticles detected in a sample from a second subject that does not have the indeterminate form of Chagas disease. As an alternative to obtaining a second sample a reference value determined using one or more subjects who do not have the indeterminate form of Chagas disease may also be used.

In certain non-limiting embodiments, the electron dense microparticles are electron dense lipidic particles.

In certain non-limiting embodiments, the second subject does not have Chagas disease.

In certain embodiments, the second subject has Chagasic cardiomyopathy.

In certain non-limiting embodiments, the electron dense microparticles have a diameter that is less than about 100 nm, less than about 75 inn, less than about 50 nm, less than about 25 nm, less than about 10 nm, less than about 5 nm, or less than about 1 nm.

In certain non-limiting embodiments, the electron dense microparticles have a diameter that is between about 0.5 and about 100 nm, or between about 1 and about 75 nm, or between about 5 and about 50 nm, or between about 10 and about 25 nm.

In certain non-limiting embodiments, the electron dense microparticles are associated with Mycoplasma pneumoniae antigens. In certain non-limiting embodiments, the concentration or number of electron dense microparticles associated with Mycoplasma pneumoniae antigens in the sample from the first subject is greater than the concentration or number of electron dense microparticles associated with Mycoplasma pneumoniae antigens in the sample from the second subject.

In certain non-limiting embodiments, the electron dense microparticles comprise exosomes.

In certain non-limiting embodiments, the first and second subject samples are independently selected from the group consisting of serum, blood, plasma and endomyocardial biopsy.

In certain non-limiting embodiments, the present invention provides for a method for diagnosing Chagasic cardiomyopathy in a subject diagnosed with Chagas disease comprising obtaining a first sample from the subject; obtaining a second sample from a second subject diagnosed with Chagas disease, wherein the second subject does not have Chagasic cardiomyopathy; detecting the presence of electron lucent microparticles in the first and second samples, wherein the presence of a greater number of electron lucent microparticles in the first sample compared to the second sample indicates a diagnosis of Chagasic cardiomyopathy. As an alternative to obtaining a second sample a reference value determined using one or more subjects who do not have Chagasic cardiomyopathy may also be used.

In certain non-limiting embodiments, the electron lucent microparticles in the first sample have smaller diameters compared to the particles detected in the second sample.

In certain non-limiting embodiments, the first sample comprises a lower concentration of nucleic acid compared to the second sample.

In certain non-limiting embodiments, the electron lucent microparticles comprise nucleic acid within the particles, and wherein the number of particles detected in the first sample is positively correlated with the presence of nucleic acid external to the particles.

In certain non-limiting embodiments, each sample is independently selected from the group consisting of serum, blood, plasma and cell culture of a cancer cell sample.

In certain non-limiting embodiments, the electron lucent microparticles are associated with nucleic acid.

In certain non-limiting embodiments, the nucleic acid is archaea nucleic acid.

In certain non-limiting embodiments of the present invention, the microparticles associated with Chagasic heart failure comprise a lower concentration of nucleic acid than the particles associated with the indeterminate form of Chagas disease.

In certain non-limiting embodiments of the present invention, the microparticles associated with the indeterminate form of Chagas disease comprise archaeosomes.

In certain embodiments, detecting a greater number of microparticles (e.g., electron dense microparticles as described herein) which comprise protein, for example, collagenase, such as, but not limited to, archaea collagenase (e.g., Archaemetzincin-1, Archeobacterial metalloproteinase-like protein 1, AMZ1) in a sample from a first subject compared to a sample from a second subject with heart failure associated with Chagas disease, indicates a diagnosis of the indeterminate form of Chagas disease in the first subject.

In certain embodiments, detecting a greater amount or level of protein, for example, collagenase, such as, but not limited to, archaea collagenase (e.g., Archaemetzincin-1, Archeobacterial metalloproteinase-like protein 1, AMZ1) external to microparticles (e.g., electron dense microparticles as described herein) in a sample from a first subject compared to a sample from a second subject with the indeterminate form of Chagas disease, indicates a diagnosis of heart failure associated with Chagas disease in the first subject.

### 5.2 Therapeutic Compositions

The present invention provides for compositions and methods that prevent or treat diseases associated with undesirable cell proliferation and fibrosis. For example, the compositions and methods of the invention inhibit the narrowing of blood vessels and reduce atherosclerosis. The compositions and methods of the present invention also decrease the level of total serum cholesterol as well as serum LDL, serum HDL and triglyceride levels in a treated patient.

In certain embodiments, administration of the compounds of the invention has the effect of reducing the presence of atherosclerotic plaques on a blood vessel, and decreasing the level of one or more of blood serum lipids, total serum cholesterol, serum LDL, serum HDL, and/or triglycerides of a treated individual.

The present invention also provides for compositions and methods that prevent or treat cancer and cardiotoxicity, heart disease or heart failure in cancer patients undergoing treatment for cancer. For example, the compositions and methods of the invention inhibit or reduce symptoms of cardiovascular toxicity, for example, QT prolongation and arrhythmias, myocardial ischemia and infarction, hypertension, venous and arterial thrombo-embolism, cardiac dysfunction, heart failure or combinations thereof.

The present invention also provides for compositions and methods that prevent or treat heart disease, for example, cardiomyopathies resulting from Chagas disease. For example, the compositions and methods of the invention inhibit or reduce the occurrence of dilated cardiomyopathy and heart rhythm abnormalities.

In particular embodiments of the invention, the composition comprises a protein capable of removing sialic acid residues, wherein removal of the sialic acid residues inhibits or prevents the attachment of a mycoplasma and one or more non-mycoplasma microorganism to a host cell. Preferred non-limiting embodiments further comprise a metal chelator and/or one or more purified plant extracts.

The term “composition” as used herein means agents or mixtures or combinations thereof effective to prevent or reduce the ability of the mycoplasma and non-mycoplasma to associate with a substrate, for example, but not limited to, a blood vessel. In certain embodiments, the composition reduces the amount of an agent, for example, a microbe or microbe nucleic acid, present in the serum, blood or plasma of a subject. In certain embodiments, the composition reduces the amount of an agent, for example, a microbe or microbe nucleic acid, present in a cell culture of a sample from a patient, for example, a cancer cell sample.

In certain embodiments, the composition reduces or inhibits the rate of growth of an atherosclerotic lesion and/or to decreases the presence of a mycoplasma and non-mycoplasma microorganism with an atherosclerotic plaque. In certain embodiments, the composition inhibits the association of a mycoplasma and a non-mycoplasma microorganism. In certain embodiments, the composition reduces or inhibits the presence of pathogenic archaea and/or microbes in the serum, blood or plasma of a subject administered the composition. In certain embodiments, the composition reduces the symptoms of cardiomyopathies resulting from Chagas disease. In certain embodiments, the composition reduces the presence of cancer cells in a subject treated with the composition.

The term “atherosclerosis,” “atherosclerotic plaque,” “plaque,” or “atheroma” as used herein refers to the accumulation of one or more of lipids, cholesterol, collagen, and macrophages on the walls of a subject's blood vessel. The presence of plaques in a blood vessel can also be associated with ossification and calcification of the blood vessel walls.

The term “blood serum lipids” as used herein refers to HDL and LDL lipoproteins.

The term “HDL” as used herein means high density lipoprotein. The term “LDL” as used herein means low density lipoprotein. In further non-limiting embodiments of the invention, the mycoplasma may be Mycoplasma (M.) buccale, M. faucium, M. fermentans, M. genitalium, M. hominis, M. lipophilum, M. oral, M. penetrans, M. pneumoniae, M. salivarium, or M. spermatophilum, wherein the mycoplasma is associated with one or more additional non-mycoplasma microorganisms. The one or more additional non-mycoplasma microorganism may be a bacteria, archaea or virus, for example, but not limited to, spirochete or chlamydia such as Chlamydia pneumoniae. According to the invention, the mycoplasma and non-mycoplasma may be attached to a substrate, for example, but not limited to, a blood vessel or an atherosclerotic plaque. In a further non-limiting embodiment, the mycoplasma and non-mycoplasma are attached to the substrate by sialic acid. In other embodiments, the mycoplasma and/or non-mycoplasma may be present in the serum, blood or plasma of a subject. In certain embodiments, the mycoplasma and/or non-mycoplasma may be present in a cell culture of a sample from a subject, for example, a cancer cell sample.

In a preferred embodiment of the invention, the protein capable of removing sialic acid residues is a trans-sialidase or neuraminidase enzyme A combination of such enzymes or an enzyme having both activities may also be used.

In certain non-limiting embodiments, the composition comprises a neuraminidase enzyme of, for example but not limited to, Bacteroides fragilis, Streptococcus pneumoniae, Streptococcus oralis, Arthrobacter ureafaciens, Clostridium perfringens, Mycoplasma alligatoris, Arcanobacterium pyogenes, Clostridium sordellii, Pseudomonas aeruginosa, Micromonospora viridifaciens, Vibrio cholerae. Streptomyces avermitilis, Influenza virus, Streptomyces coelicolor, Flavobacteriales bacterium, and Solibacter usitatus.

In other non limiting embodiments, the protein is a trans-sialidase, for example, the trans-sialidase enzyme of Trypanosoma brucei.

In a preferred embodiment, the composition is the trans-sialidase enzyme of Trypanosoma cruzi, or a portion or variant of the native enzyme which has trans-sialidase activity.

Alternatively, the trans-sialidase enzyme can be a recombinant trans-sialidase enzyme.

In specific non-limiting embodiments, the recombinant trans-sialidase is as described in International Patent Publication WO 2002/002050 by Higuchi et al., published Jan. 10, 2002; and U.S. Pat. No. 7,108,851 by Higuchi et al., issued Sep. 19, 2006. For example, the trans-sialidase gene may be obtained from a genomic clone, isolated from a commercially available lambda Zap®II library (Stratagene, http://www.stratagene.com) of T. cruzi Y strain (Silva and Nussenzweig, 1953, Folia Clip Biol 20: 191-203), as described in Uemura et al. (Uemura et al., 1992, EMBO J 11: 3837-3844). From the original lambda clone, which expresses enzymatic activity, ate SK plasmid containing the trans-sialidase gene may be generated (SK-154-0). The preferred plasmid used is pTSII, which corresponds to a fragment of the original gene (clone 154-0) amplified through PCR, and inserted into the sites NdeI and BamHI of the vector pET14b (Novagen—http://www.novagen.com). The PCR product may be amplified using SK-154-0 as a template with the following primers:

(SEQ ID NO: 1)

| | |
|---|---|
| a) TSPET14: | 5′-GGAATTCCATATGGCACCCGGATCGAGC |

(SEQ ID NO: 2)

| | |
|---|---|
| b) RT154: | 5′-CGGATCCGGGCGTACTTCTTTCACTGGTGCCGGT |

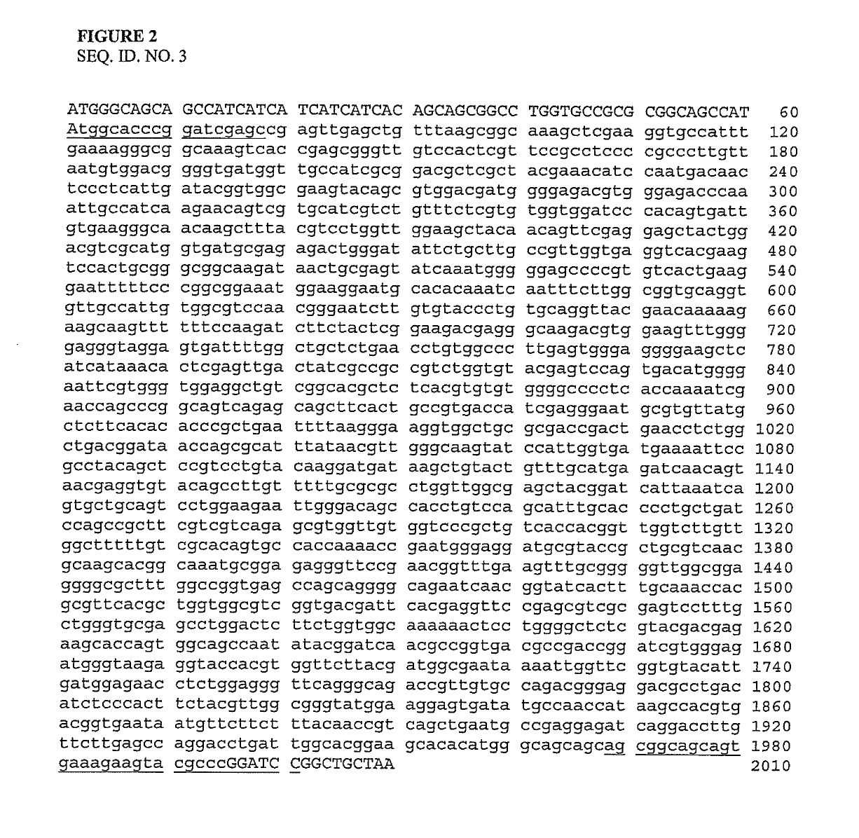

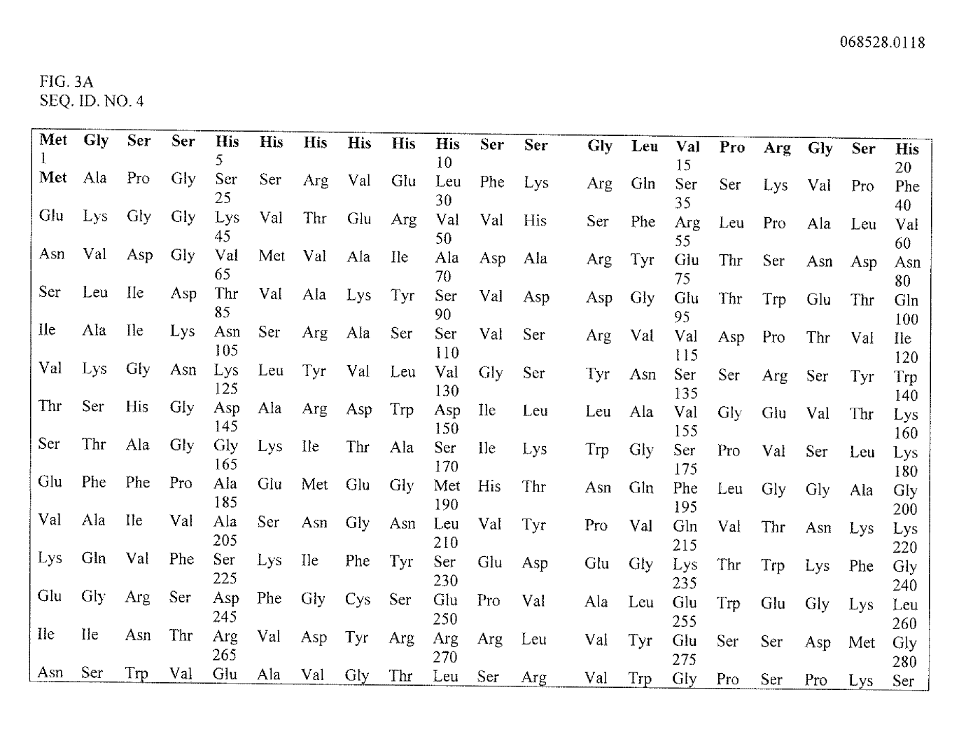

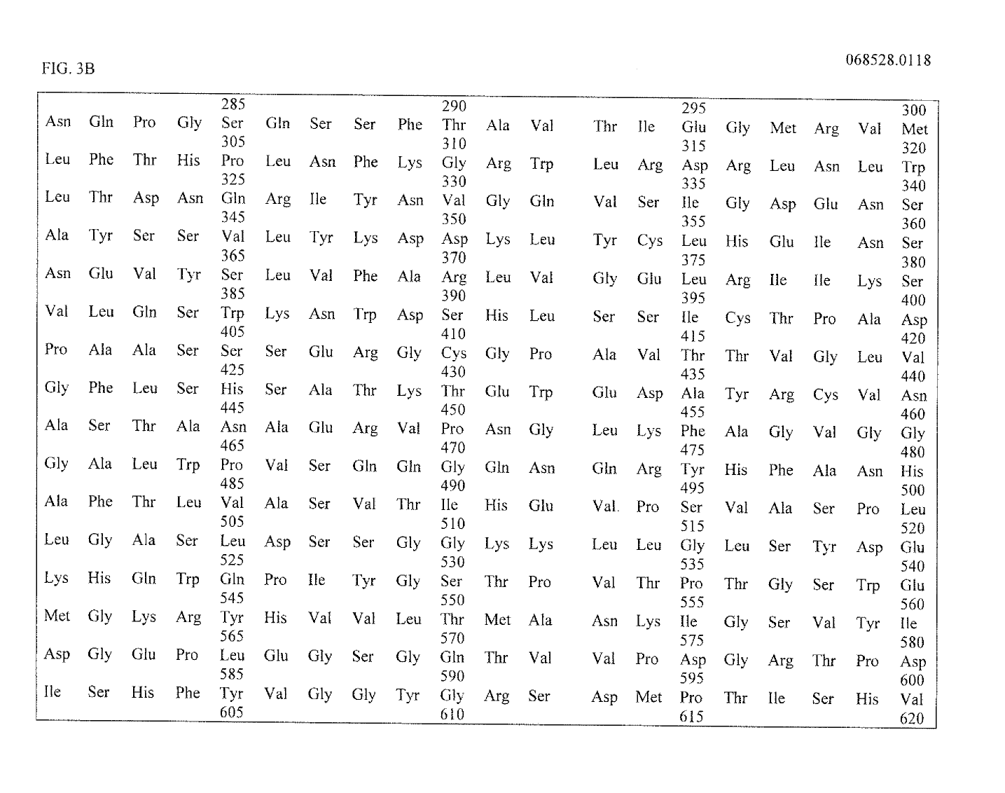

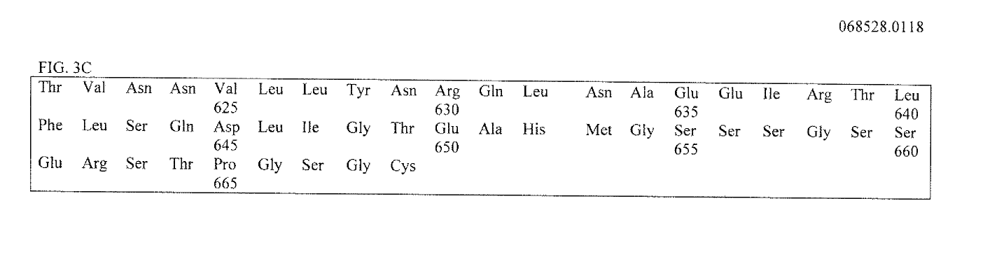

The resulting PCR product should have a nucleic acid sequence as set forth in FIG. 2 (SEQ ID NO:3), and a corresponding amino acid sequence as depicted in FIG. 3 (SEQ ID NO:4). The resulting plasmid may be transformed into the Escherichia coli BLB21 DE3. The construct can be made in two steps due to an internal BamHI site in the trans-sialidase gene. The PCR product may be treated with BamHI and NdeI enzymes, and the resulting fragments fractionated by electrophoresis on an agarose gel. The separated fractions may then be purified from the gel with the Sephaglass purification kit (Amersham-Pharmacia). The 5′ NdeI-BamHI digestion fragment may be ligated into the pET14b vector which has been pre-digested with BamHI and NdeI. The ligation products may be used to transform K12 DH5a E. coli cells. The plasmid containing E. coli cells may be selected and the plasmid purified by methods known in the art. The purified construct may be treated with BamHI, shrimp alkaline phosphatase, and ligated with the BamHI-BamHI-3′ fragment purified from the fractionation gel. The ligation products may then be used to transform K12 DH5a E. coli cells, from which clones expression of trans-sialidase may be selected and purified. The final plasmid may be confirmed by restriction analysis and used to transform the BLB21 DE3 pLys strain of E. coli, from which recombinant trans-sialidase enzyme can be purified, as described in International Patent Publication WO/2002/002050 by Higuchi et al., published Jan. 10, 2002; and U.S. Pat. No. 7,108,851 by Higuchi et al., issued Sep. 19, 2006.

Alternatively, the trans-sialidase enzyme may be purified from a culture of Trypanosoma cruzi, such as, for example, a culture according to Kloetzel et al. (Kloetzel et al., 1984, Rev. Inst. Med. Trop. Sao Paulo., 26:179-85). Supernatant from the culture may be filtered through a 1 μm pore filter in a vacuum chamber. The enzyme may be further purified by filtering the supernatant through a 0.22 μm filter and then precipitating the filtrate with a 50% (NH₄)2SO₄ solution. The precipitates may then be dialyzed against phosphate-buffered saline, and passed through a tresyl-agarose column comprising an immobilized anti-trans-sialidase monoclonal or polyclonal antibody. The column may be washed with phosphate-buffered saline, followed by an additional wash with 10 mM sodium phosphate, pH 6.5. The trans-sialidase may then be eluted with a 3.5 mM MgCl₂, 10 mM sodium phosphate, pH 6.0 solution. The fractions eluted from the column may be filtered through a Sephadex G-25 column equilibrated with 20 mM Tris-HCl, pH 8.0, to remove the MgCl₂. The trans-sialidase may be further purified by passage through a Mono Q column equilibrated in 20 mM Tris-HCl, pH 8.0, and eluted with a linear gradient from 0 to 1 mM NaCl in the same buffer.

The purified enzyme derived from the culture should comprise 400 kDa multimeric aggregates. The enzymatic activity of the purified trans-sialidase may be measured according to methods described in International Patent Publication WO 2002/002050 by Higuchi et al., published Jan. 10, 2002; and U.S. Pat. No. 7,108,851 by Higuchi et al., issued Sep. 19, 2006.

In non-limiting embodiments, the purified trans-sialidase has an enzymatic activity of between 0.1 and 10 U/ml, more preferably between 1.0 and 5.0 U/ml, and most preferably 1.3 U/ml.

In certain non-limiting embodiments, the composition comprises a metal chelator, for example, but not limited to, Nitrilotriacetate (NTA), diphenylthiocarbazone(dithizone), histidine, the lipophilic metal chelator DP-109, ethylene glycol tetraacetic acid (EGTA), ethylenediaminetetraacetic acid (EDTA), DMPS (2,3-dimercapto-1-propanesulfonate), Lysinoalanine, Synthetic lysinoalanine (N-ε-DL-(2-amino-2-carboxyethyl)-L-lysine), tetracycline, alpha lipoic acid (ALA), Dimercaptosuccinic acid, (DMSA), 2,3-Dimercapto-1-propanesulfonic acid (DMPS), Calcium disodium versante (CaNa₂-EDTA), D-penicillamine, Deferoxamine, Defarasirox, Dimercaprol (BAL), the calcium salt of diethylene triamine pentaacetic acid (DTPA), or any other metal chelator known in the art. In a preferred non-limiting embodiment, the metal chelator is pyrrolidine dithiocarbamate (PDTC). The composition of the invention may comprise the metal chelator in a concentration of between about 0.01 and 10 mg/ml, more preferably between about 0.5 and 5 mg/ml, more preferably between about 1 and 2 mg/ml, and most preferably about 1.5 mg/ml.

In a further non-limiting embodiments, the plant extract may be derived from, for example but not limited to, Allium sativum (garlic), Ginkgo biloba, tomato, orchid, guava, ginseng, for example Pfaffia paniculata (Brazilian ginseng); Zingiber officinale (ginger); or tobacco, wherein the orchid is preferably of the genus Cymbidium, for example, yellow or green orchids from the genus Cymbidium (Cymbidium ssp). Alternatively, the orchid may be of the genus Dendrobium, for example, Dendrobium nobile or Dendrobium moschatum.

The extract from plants may be obtained by adding a solvent, such as, for example, alcohol, to the plant tissue, for example, but not limited to, roots, cloves, flower petals, or leaves which may be chopped, or macerated prior to mixture with the solvent. The solvent may be mixed with the plant tissue in a proportion of between 1:99 and 60:40, more preferably between 15:85 and 50:50 and most preferably between 30-40:70-60 of plant mass:alcohol. The solvent can be an alcohol, for example, ethanol, methanol, or grain alcohol, and can have a concentration of between 60% and 100%, more preferably between 70% and 95%, and most preferably 92% alcohol. The plant/alcohol mixture may be aged in a dark, anaerobic environment for a period of time between 15 days and 24 months, more preferably between 1 and 15 months, and most preferably 12 months.

According to the invention, the extract derived from plant comprises particles containing nucleic acid (DNA or RNA), wherein the particle is an archaea (preferably non-pathogenic) and/or a nanoarchaea, and further wherein the particle is present in an amount effective to prevent or inhibit the growth of a mycoplasma and one or more non-mycoplasma microorganisms. Aging of the plant/alcohol mixture increases the concentration of particles in the mixture.

In certain embodiments, the non-pathogenic archaea and/or nanoarchaea comprise protein, for example, collagenase, such as but not limited to archaea collagenase (e.g., Archaemetzincin-1, Archeobacterial metalloproteinase-like protein 1, AMZ1).

The plant/alcohol mixture may be purified, and the concentration of nanoparticles may be increased through one or more filtrations. The mixture may be filtered through pores of between 0.5 μm and 50 μm, more preferably between 5 μm and 20 μm, and most preferably for example, but not limited to Whatman qualitative filter paper grade 1, diameter 24 cm, pore size 11 μM. Vacuum chambers can also be used separately, or in addition to other filtration methods. Additionally, glass microfiber filters may be used for filtration, for example, but not limited to, a 47 mm diameter glass microfiber filter with a pore size of 1.1 μm. Any filtration methods known in the art may be used to filter the aged plant/alcohol mixture.

In a non-limiting embodiment, the plant/alcohol mixture can be subjected to additional aging during the filtration process. For example, olive oil may be added to the filtrate to create a 1% olive oil filtrate mixture, followed by an additional month of storage in a dark anaerobic environment.

According to the methods of the present invention, aging a plant extract increases the proportion of non-pathogenic archaea to pathogenic archaea in the plant extract. In certain embodiments, the plant extracts are prepared in ethanol solution, followed by filtration in filter with pores of 0.4 um, then aging in 0.12% of extra virgin olive oil, and filtration again to extract the oil.

In one embodiment, aging the plant extract increases the number of non-pathogenic archaea in the plant extract.

In another embodiment, aging the plant extract decreases the number of pathogenic archaea in the plant extract.

In another embodiment, an aged plant extract, or alternatively, a plant extract that has not been aged, can be diluted with a dilutant and aged for an additional period of time.

In a further non-limiting embodiment, the dilutant can be thermal water, oil, for example, olive oil, or any other dilutant known in the art.

In another non-limiting embodiment, the plant extract or the diluted plant extract can be aged for between 15 days and 24 months.

In another non-limiting embodiment, the plant extract or the diluted plant extract, can be aged for 30 days.

In certain embodiments, the plant extract is prepared from olive oil. In certain embodiments, the plant extracts are prepared from olive oil as described by Gonzalez-Paredes et al., International Journal of Pharmaceuticals, 421 (2011) 321-331, which is incorporated by reference in its entirety herein.

Furthermore, the composition may comprise particles and/or nanoparticles containing DNA or RNA, wherein the particles are a non-pathogenic archaea and/or a nanoarchaea, and further wherein the particle is present in amounts effective to prevent or inhibit the growth of a mycoplasma and one or more non-mycoplasma microorganisms. The nanoparticles may be between 5-500 nm, more preferably between 15-250 mm, and most preferably between 30-150 nm in diameter. Alternatively, the composition may comprise medium particles of between 500 nm and 1.1 μm in diameter. Additionally, the compositions may comprise one or a combination of both small and medium particles. The size of a particle can enlarge or decrease depending on the concentration of water and ions in a solution comprising the particles, such as, for example, Na+ or Ca+.

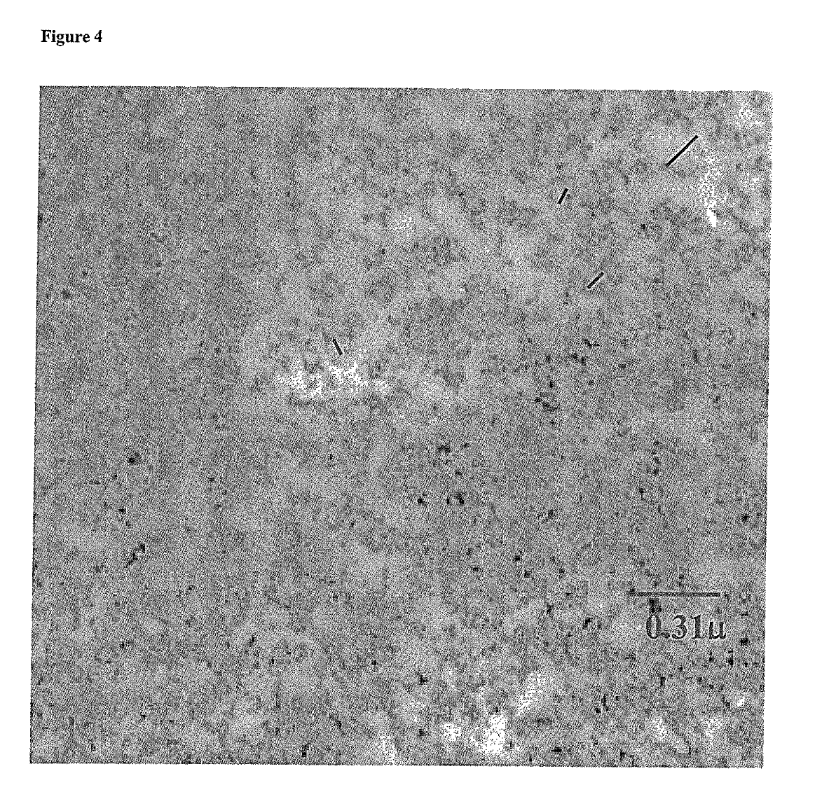

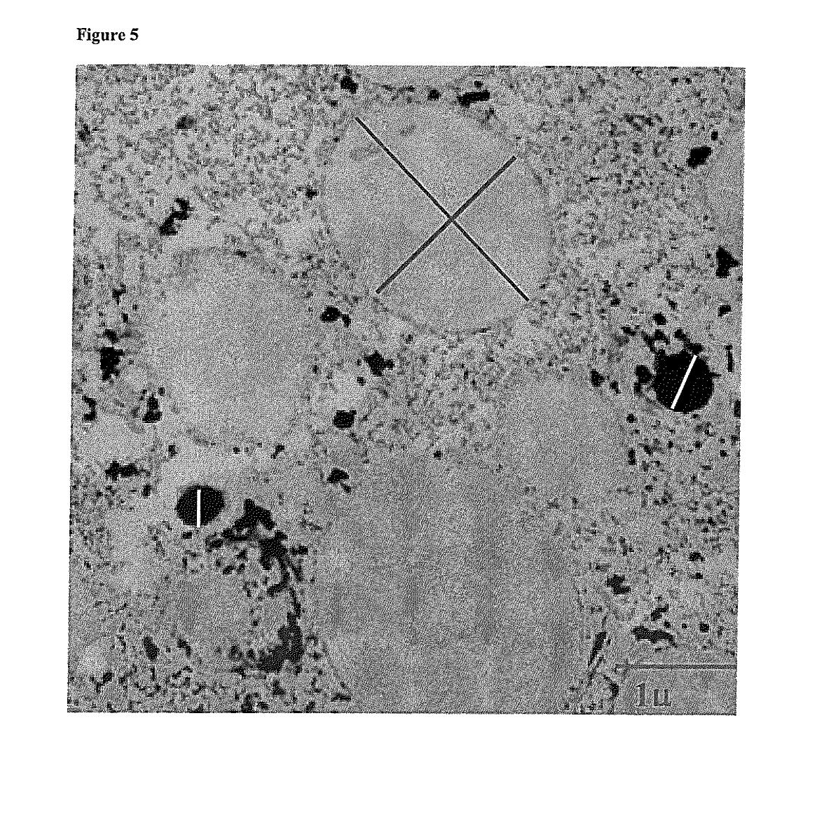

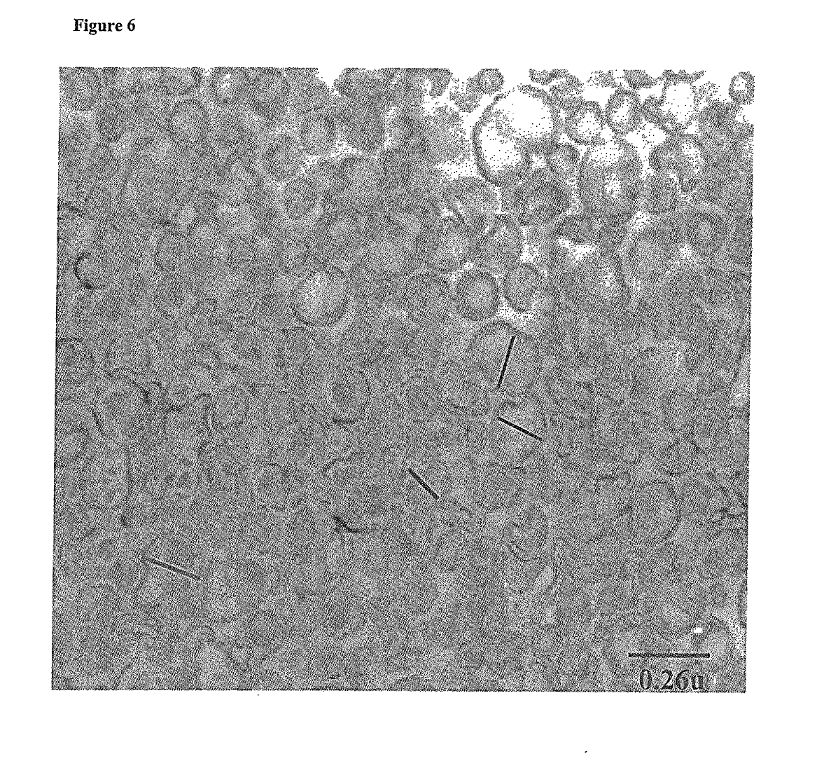

According to the invention, the purity of the plant extract may be determined by microscopic examination of the filtered, aged, plant extract, as described in U.S. Patent Application Publication No. 20050142116. For example, the filtered, aged plant extract can be stained with any DNA or RNA dye known in the art, such as acridine orange, bisbenzimide H 33342 (Hoechst), or 4′,6-diamidino-2-phenylindole, dihydrochloride (DAPI); and viewed with an immunofluorescence optical microscope, an electron microscope, or any other microscope known in the art. Two forms of archaea, having different morphological characteristics may be identified. One type comprising an electron-dense content may be between about 0.03-0.15 μm (nanoparticle) and about 0.5-1.1 μm in diameter (medium particle) (FIGS. 4 and 5, respectively). A second type may comprises a clear, empty content, and may be about 0.15-2.4 μm in diameter (FIGS. 5 and 6). The clear, empty archaea are similar in morphology to the pathogenic archaea associated with lesions, while the electron dense archaea comprise the non-pathogenic archaea and nanoarchaea comprising DNA or RNA. Brilliant red particles, which may comprise metallic ions, may also adhere to the surface of the archaea. Optimum purity may be achieved when predominantly, preferably essentially, only fast moving electron-dense nanoparticles are visible. The presence of clear, empty archaea or large brilliant red particles of about 0.15-0.24 μm and at a concentration of, for example, ≧1.0 large brilliant red particle/visual field, indicates suboptimal purity. In cases of suboptimal purity, the filtered aged plant extract is subjected to additional filtration, for example, tangential flow filtration in the Minitan Ultrafiltration System (Millipore, Bedford, Mass., USA), using the macroporous membrane packet (30,000 NMWL). In preferred embodiments, the compositions of the invention comprise a greater number of electron dense archaea (nanoparticles and medium particles) than empty, clear archaea; and a greater number of archaea not associated with large brilliant red particles than those associated with large brilliant red particles.

According to the invention, the purified plant extract may comprise an enriched population of particles. The concentration of particles may be between 1×10⁵ and 1×10¹⁰ particles/ml, more preferably between 1×10⁶ and 1×10⁹ particles/ml, and most preferably about 1×10⁷ particles/ml.

In a non-limiting embodiment, the compositions of the invention comprise combinations of trans-sialidase, a metal chelator, and one or more purified plant extracts as shown in Table I.

TABLE I
Combinations of trans-sialidase, a metal chelator, and one or more
purified plant extracts encompassed by the invention.
Combinations of trans-sialidase (TS), pyrrolidine dithiocarbamate
(PDTC), and purified plant extracts

TS
TS + PDTC
TS + PDTC + Allium sativum (AS)
TS + PDTC + Ginkgo biloba (GB)
TS + PDTC + Zingiber officinale (ZO)
TS + PDTC + orchid extract (OE)
TS + PDTC + AS + GB
TS + PDTC + AS + ZO
TS + PDTC + AS + OE
TS + PDTC + AS + GB + ZO
TS + PDTC + AS + GB + OE
TS + PDTC + AS + GB + ZO + OE
TS + PDTC + AS + ZO + OE
TS + PDTC + GB + ZO
TS + PDTC + GB + OE
TS + PDTC + GB + ZO + OE
TS + PDTC + ZO + OE
TS + AS
TS + GB
TS + ZO
TS + OE
TS + AS + GB
TS + AS + ZO
TS + AS + OE
TS + AS + GB + ZO
TS + AS + GB + OE
TS + AS + GB + ZO + OE
TS + AS + ZO + OE
TS + GB + ZO
TS + GB + OE
TS + GB + ZO + OE
TS + ZO + OE

### 5.3 Therapeutic Uses

The present invention provides for compositions and methods for reducing the presence of atherosclerotic plaques in a blood vessel. The compositions and methods of the invention further provide for reducing the level of total serum cholesterol in a treated subject, as well as serum LDL, HDL and triglyceride levels.

In certain embodiments, the present invention provides for compositions and methods for inhibiting or reducing symptoms of cardiotoxicity, heart disease or heart failure in cancer patients undergoing treatment for cancer, for example, QT prolongation and arrhythmias, myocardial ischemia and infarction, hypertension, venous and arterial thrombo-embolism, cardiac dysfunction, heart failure or combinations thereof.

In certain embodiments, the present invention provides for compositions and methods for treating cancer in a subject, for example, by inhibiting or reducing the presence of cancer cells in a subject.

In certain embodiments, the present invention provides for compositions and methods for preventing or treating heart disease, for example, cardiomyopathies resulting from Chagas disease, for example, inhibiting or reducing the occurrence of dilated cardiomyopathy and heart rhythm abnormalities.

In certain embodiments, the composition of the invention comprises a trans-sialidase enzyme, PDTC, and one or more purified plant extracts.

In one embodiment, the composition of the invention may be administered in an amount effective to reduce the presence of an atherosclerotic plaque.

In other embodiments, the composition of the invention may be administered in an amount effective to inhibit or reduce QT prolongation, heart arrhythmias, myocardial ischemia, myocardial infarction, hypertension, venous and/or arterial thrombo-embolism, cardiac dysfunction, the occurrence of dilated cardiomyopathy, heart failure or combinations thereof.

In certain embodiments, the composition of the invention is administered to a subject diagnosed with Chagas disease or with cardiomyopathies resulting from Chagas disease.

In certain embodiments, the composition of the invention is administered to a subject diagnosed with cancer. In certain embodiments the subject is a cancer patient receiving treatment for cancer, for example, chemotherapy. In certain embodiments, the cancer patient receiving cancer treatment has been diagnosed has having cardiotoxicity, heart disease or heart failure.

In a non-limiting embodiment of the invention, the composition may be administered systemically, for example, as an injection. In another preferred embodiment of the invention, the composition may be administered orally. According to the invention, the composition is effective to promote a reduction in the presence of one or more mycoplasma and one or more non-mycoplasma microorganism on a blood vessel wall as compared to a subject not treated with the composition. For example, the presence of Mycoplasma pneumoniae and Chlamydia pneumoniae is reduced in atherosclerotic plaques.

In certain embodiments, the composition is effective to promote a reduction in the presence of one or more agent, for example, a microbe or microbe nucleic acid, in the serum, blood or plasma as compared to a subject not treated with the composition.

In certain embodiments, the composition is effective to promote a reduction in the presence of one or more agent, for example, a microbe or microbe nucleic acid, in cell culture of a sample, for example, a cancer cell sample from a subject, as compared to a cell culture not treated with the composition.

In another series of non-limiting embodiments, the composition may be administered as a single dose, or at regular intervals so that the composition is effective to promote a reduction in the presence or level of atherosclerotic plaques, total serum cholesterol, serum LDL, serum HDL, triglyceride, QT prolongation, heart arrhythmias, myocardial ischemia, myocardial infarction, hypertension, venous and/or arterial thrombo-embolism, cardiac dysfunction, the occurrence of dilated cardiomyopathy, heart failure or combinations thereof, in a subject as compared to a subject not treated with the composition.

In a non-limiting embodiment of the invention, the composition may be administered in an amount effective to reduce the surface area of a blood vessel covered by an atherosclerotic plaque. The composition may decrease the percentage of a blood vessel's surface area occupied by a plaque to between about 0% and 75%, more preferable between 2% and 50%, more preferably between 5% and 60%, more preferably between 10% and 25% and most preferably about 11% of the total surface area of the blood vessel.

In another non-limiting embodiment of the invention, the composition may be administered in an amount effective to reduce the level of total serum cholesterol in a subject in need of treatment. The composition may reduce the level of total serum cholesterol of the subject by about 5%, 10%, 20%, 50%, 90% or 95% such that the level of total cholesterol is reduced to about the normal level found in a subject not in need of treatment.

In another non-limiting embodiment of the invention, the composition may be administered in an amount effective to reduce the level of serum LDL cholesterol in a subject in need of treatment. The composition may reduce the level of serum LDL cholesterol of the subject by about 5%, 10%, 20%, 50%, 90% or 95% such that the level of serum LDL cholesterol is reduced to about the normal level found in a subject not in need of treatment.

In another non-limiting embodiment of the invention, the composition may be administered in an amount effective to reduce the level of serum HDL cholesterol in a subject in need of treatment. The composition may reduce the level of serum HDL cholesterol of the subject by about 5%, 10%, 20%, 50%, 90% or 95% such that the level of serum HDL cholesterol is reduced to about the normal level found in a subject not in need of treatment.

In another non-limiting embodiment of the invention, the composition may be administered in an amount effective to reduce the level of triglycerides in a subject in need of treatment. The composition may reduce the level of triglycerides of the subject by about 5%, 10%, 20%, 50%, 90% or 95% such that the level of triglycerides is reduced to about the normal level found in a subject not in need of treatment.

In a further non-limiting embodiment of the invention, the normal level of total serum cholesterol is about 200 mg/dl or less, the normal level of serum LDL cholesterol is about 100 mg/dl or less, the normal level of serum HDL cholesterol is about 60 mg/dl or more, and the normal level of triglycerides is about 150 mg/dl or less (American Heart Association website, Jan. 30, 2007).

In another non-limiting embodiment of the invention, the composition may be administered in an amount effective to reduce the presence of one or more microorganism with an atherosclerotic plaque, for example, but not limited to Mycoplasma pneumoniae and Chlamydia pneumoniae, wherein the reduction in microorganism presence is indicated by a reduction in the detection of the microorganisms' antigens. According to the invention, the reduction in antigen detection is between about 0.1 and 100%, and most preferably 99% as compared to the antigen detection in an untreated subject.

In another non-limiting embodiment of the invention, the composition may be administered in an amount effective to reduce the amount of an agent, for example a microbe or microbe nucleic acid, in the serum, blood or plasma of a subject in need of treatment. The composition may reduce the amount of agent in the serum, blood or plasma by about 5%, 10%, 20%, 50%, 90% or 95% such that the amount of agent is reduced to about the normal level found in a subject not in need of treatment.

In another non-limiting embodiment of the invention, the composition may be administered in an amount effective to reduce the amount of an agent, for example a microbe or microbe nucleic acid, in a cell culture of a sample, for example, a cancer cell sample, from a subject in need of treatment. The composition may reduce the amount of agent in the cell culture by about 5%, 10%, 20%, 50%, 90% or 95% such that the amount of agent is reduced to about the normal level found in a subject not in need of treatment.

The composition may be administered locally or systemically, for example, by injection, orally, occularly, rectally, topically, or by any other means known in the art. The composition may be ingested as a liquid, a pill, or a capsule (e.g. liquid or powder-filled).

In one non-limiting embodiment, the composition may comprise a trans-sialidase, a metal chelator, for example, but not limited to, PDTC, NTA, diphenylthiocarbazone(dithizone), histidine, DP-109, EGTA, EDTA, DMPS, Lysinoalanine, Synthetic lysinoalanine, tetracycline, ALA, Dimercaptosuceinic acid, DMSA, Calcium disodium versante, D-penicillamine, Deferoxamine, Defarasirox, Dimercaprol, and DTPA; and one or more purified plant extract. The trans-sialidase may have an enzymatic activity of between about 0.01 and 10 U/ml, more preferably between about 0.2 and 5 U/ml, more preferably between about 0.5 and 2 Uml and most preferably about 1.0 U/ml. The metal chelator may have a concentration of between about 0.01 and 10 mg/ml, more preferably between about 0.5 and 5 mg/ml, more preferably between about 1 and 2 mg/ml, and most preferably 1.5 mg/ml. The purified plant extract may comprise a particle concentration of between about 1×10⁵ and 1×10⁷ particles/ml, more preferably between about 5×10⁶ and 9×10⁶ particles/ml, more preferably between about 2×10⁶ and 3×10⁶ particles/ml, and most preferably about 1.0×10⁶ particles/ml.

In a specific non-limiting embodiment, the composition is administered as an injection, wherein the composition comprises a trans-sialidase, PDTC and one or more purified plant extract, further wherein the trans-sialidase has an enzymatic activity of 1.04 U/ml, the PDTC has a concentration of 1.5 mg/ml, and the purified plant extract has a particle concentration of 1.0×10⁶ particles/ml.

In an alternative non-limiting embodiment, the composition may comprise a trans-sialidase, a metal chelator, and one or more purified plant extract, wherein the trans-sialidase comprises an enzymatic activity of between about 1×10⁻⁸ and 1×10⁻⁴ U/ml, more preferably between about 1×10⁻⁷ and 1×10⁻⁵ U/ml, more preferably between about 1×10⁻⁶ and 5×10⁻⁶ U/ml and most preferably about 1.5×10⁻⁶ U/ml. The metal chelator may have a concentration of between about 0.01 and 10 mg/ml, more preferably between about 0.5 and 5 mg/ml, more preferably between about 1 and 2 mg/ml, and most preferably 1.5 mg/ml. The purified plant extract may comprise a particle concentration of between about 1×10⁵ and 1×10⁷ particles/ml, more preferably between about 2×10⁶ and 9×10⁶ particles/ml, more preferably between about 3×10⁶ and 7×10⁶ particles/ml, and most preferably about 5×10⁶ particles/ml.

In a specific non-limiting embodiment, the composition is administered orally as a liquid, wherein the composition comprises a trans-sialidase and one or more purified plant extract, further wherein the trans-sialidase has an enzymatic activity of 1.3×10⁻⁶ U/ml and the purified plant extract has a particle concentration of 5.0×10⁶ particles/ml.

In another non-limiting embodiment, the composition is administered in an amount of between 0.002 and 5.0 nil/kg, more preferably between 0.1 and 2.0 ml/kg, more preferably between 0.2 and 1.0 ml/kg, and most preferably about 0.25-0.5 ml/kg.

In a further non-limiting embodiment, the composition may be administered once, twice, three, four, five, or six or more times per day during the treatment period. Alternatively, the composition may be administered once every two, three, four, five, six or seven or more days.

In a non-limiting example of the invention, the composition is a mixture of trans-sialidase and PDTC, wherein the trans-sialidase has an activity of about 1.04 U/ml and the PDTC is at a concentration of 1.5 mg/ml, and wherein the composition is administered via intraperitoneal or intravenous injection at a volume of about 25-0.5 ml/kg every other day.

In a further non-limiting example of the invention, the mixture of trans-sialidase and PDTC is supplemented with a purified plant extract diluted 1:10 in purified water, and containing an average of 1.0×10⁶ nanoparticles/ml. The plant extract dilution is administered through intraperitoneal injections once per day for a four week treatment period. Examples of mixtures include, but are not limited to, TS+PDTC, TS+PDTC+AS extract, TS+PDTC+AS+GB extracts, and TS+PDTC+AS+GB+ZO extracts. For each of the mixtures, the TS+PDTC may be injected intravenously or ingested orally in an amount of 0.25-0.5 ml/kg every other day during a 12 week treatment session, wherein the mixture comprises 1.04 U/ml TS activity and 1.5 mg/ml PDTC. Each of the plant extracts comprise a 1:10 plant extract:water dilution which further comprise 1.0×10⁶ nanoparticles/ml. A total volume of 1 ml of diluted plant extract is injected intraperitoneally once daily during the 12 week treatment period. When more than one diluted plant extract is used, the different extracts are mixed in equal volumes.

In other non-limiting embodiments of the invention, success of treatment for atherosclerosis and/or a mycoplasma associated disease, cardiotoxicity, heart disease or heart failure in a cancer patient receiving treatment for cancer, or Chagas disease cardiomyopathy can be determined by detecting archaea and/or mycoplasma and/or chlamydia and/or spirochete forms or their products in the serum or atherosclerotic lesions of a patient. In one non-limiting embodiment, a decrease in the number of archaea and/or mycoplasma and/or chlamydia and/or spirochete forms or their products in the serum or in the atherosclerotic lesions of a patient indicates successful treatment.

In one non-limiting embodiment, a method for monitoring success of treatment for a disorder characterized by undesirable cell proliferation in a subject, cardiotoxicity, heart disease or heart failure, atherosclerosis with vulnerable plaque, heart disease or heart failure in a cancer patient receiving treatment for cancer, or Chagas disease cardiomyopathy comprises obtaining a first sample from the subject; obtaining a second sample from the subject, wherein the second sample is obtained after the first sample and after administration of the treatment; and detecting the presence of an agent (e.g., archaea and/or mycoplasma and/or chlamydia and/or spirochete) in the first and second samples, wherein a decrease in the number or concentration of agent in the second sample compared to the first sample indicates treatment success.

In one non-limiting embodiment, a method for monitoring success of treatment for a disorder characterized by undesirable cell proliferation in a subject, cardiotoxicity, heart disease or heart failure, atherosclerosis with vulnerable plaque, heart disease or heart failure in a cancer patient receiving treatment for cancer, or Chagas disease cardiomyopathy comprises obtaining a first sample from the subject; obtaining a second sample from the subject, wherein the second sample is obtained after the first sample and after administration of the treatment; and detecting the presence of an agent (e.g., electron dense microparticles comprising protein, such as collagenase, for example, archael collagenase as described herein) in the first and second samples, wherein an increase in the number or concentration of agent in the second sample compared to the first sample indicates treatment success.

In certain non-limiting embodiments, the present invention provides for methods of treating heart disease, heart failure, or atherosclerosis (for example, atherosclerosis with vulnerable plaque) in a subject comprising administering a composition to the subject comprising an agent that can remove sialic acid residues and one or more plant extracts.

In certain embodiments, the subject is in treatment for malignant neoplasia.

In certain embodiments, the plant extract comprises nucleic acid-containing particles selected from the group consisting of non-pathogenic archaea, non-pathogenic nanoarchaea, and a mixture thereof.

In certain embodiments, the plant extracts comprise microparticles comprising protein, for example, collagenase, such as, but not limited to, archaea collagenase (e.g., Archaemetzincin-1, Archeobacterial metalloproteinase-like protein 1, AMZ1).

In certain non-limiting embodiments, the subject has Chagas disease.

In certain non-limiting embodiments, the subject has vulnerable atherosclerotic plaque.

In certain non-limiting embodiments, the composition further comprises a metal chelator.

In certain non-limiting embodiments, the method of treating heart disease or heart failure further comprises monitoring success of the treatment comprising obtaining a first sample from the subject; obtaining a second sample from the subject, wherein the second sample is obtained after the first sample and after administration of the treatment; detecting the presence of an agent in the first and second samples, wherein the agent is selected from the group consisting of pathogenic archaea, pathogenic archaea nucleic acid, electron lucent microparticle, virus and combinations thereof, wherein a decrease in the number or concentration of agent in the second sample compared to the first sample indicates treatment success.

In certain non-limiting embodiments, the electron lucent microparticle is an electron lucent lipidic particle.

In certain non-limiting embodiments, the first and second samples are independently selected from the group consisting of serum, blood, plasma and endomyocardial biopsy.

In certain non-limiting embodiments, the agent is electron lucent lipidic particle.

In certain non-limiting embodiments, the electron lucent lipidic particle is associated with nucleic acid.

In certain non-limiting embodiments, the nucleic acid is archaea nucleic acid.

In certain non-limiting embodiments, the electron lucent lipidic particle comprises nucleic acid within the particle, and wherein the number of particles detected in the first sample is positively correlated with the presence of nucleic acid external to the particles.

In certain non-limiting embodiments of the present invention, the nucleic acid external to the microparticles detected according to the methods of the present invention are comprised in nanovesicles.

In certain non-limiting embodiments, the method of treating vulnerable atherosclerotic plaque in a subject further comprises monitoring success of the treatment comprising obtaining a first sample from the subject; obtaining a second sample from the subject, wherein the second sample is obtained after the first sample; detecting the presence of co-localized Mycoplasma pneumoniae and oxidized LDL in the first and second samples, wherein a decrease in the number or concentration of co-localized Mycoplasma pneumoniae and oxidized LDL in the second sample compared to the first sample indicates treatment success.

In certain non-limiting embodiments, the method of treating vulnerable atherosclerotic plaque in a subject further comprises monitoring success of the treatment comprising obtaining a first sample from the subject; obtaining a second sample from the subject, wherein the second sample is obtained after the first sample; detecting the presence of co-localized Chlamydia pneumoniae and archaea collagenase in the first and second samples, wherein a decrease in the number or concentration of co-localized Chlamydia pneumoniae and archaea collagenase in the second sample compared to the first sample indicates treatment success.

In certain non-limiting embodiments, the method of treating heart disease or heart failure further comprises monitoring success of the treatment comprising obtaining a first sample from the subject; obtaining a second sample from the subject, wherein the second sample is obtained after the first sample and after administration of the treatment; detecting the presence of an agent in the first and second samples, wherein the agent comprises electron dense microparticles comprising protein, for example, collagenase, such as, but not limited to, archaea collagenase (e.g., Archaemetzincin-1, Archeobacterial metalloproteinase-like protein 1, AMZ1), wherein an increase in the number or concentration of agent in the second sample compared to the first sample indicates treatment success.

### 5.4 In Vitro Assay

The present invention also provides for in vitro methods for selecting a composition of the present invention for use in treating a subject diagnosed with or at risk for developing a disorder characterized by undesirable cell proliferation, heart failure caused by injury or Chagas disease, dilated cardiomyopathy, cancer or cardiotoxicity during cancer treatment. In one embodiment, the in vitro method comprises assaying the effect of a composition of the present invention in reducing the presence of an agent in a sample from a patient, for example, a serum, blood or plasma sample, or a cell culture of a patient sample, for example, a cancer cell sample. In certain embodiments, the agent is a microbe or microbe nucleic acid. In one embodiment, the composition that is most effective in reducing the presence of the agent in the sample is selected for use in treating the patient.

In certain non-limiting embodiments, the present invention provides for an in vitro method for selecting a composition for use in treating a subject diagnosed with or at risk for developing heart disease or heart failure comprising contacting a first sample from the subject with a test composition; detecting the presence of an agent in the first sample, wherein the agent is selected from the group consisting of pathogenic archaea, pathogenic archaea nucleic acid, mycoplasma, chlamydia, C reactive protein, spirochete, virus, electron lucent microparticles, and combinations thereof; and detecting the presence of the agent in a second sample from the subject, wherein the second sample is not contacted with the test composition, wherein a lower concentration of agent in the first sample compared to the second sample indicates that the composition is suitable for use in treating the subject diagnosed with or at risk for developing heart disease or heart failure.

In certain non-limiting embodiments, the electron lucent microparticles are electron lucent lipidic particles.

In certain non-limiting embodiments, the agent is electron lucent microparticles, and the particles are associated with nucleic acid.

In certain non-limiting embodiments, the nucleic acid is archaea nucleic acid.

In certain non-limiting embodiments, the heart disease or heart failure is associated with Chagas disease.

In certain non-limiting embodiments, the heart disease or heart failure is associated with dilated cardiomyopathy.

In certain non-limiting embodiments, the heart disease or heart failure is cardiotoxicity resulting from cancer treatment.

In certain non-limiting embodiments, the present invention provides for an in vitro method for selecting a composition for use in treating a subject diagnosed with or at risk for developing heart disease or heart failure comprising contacting a first sample from the subject with a test composition; detecting the presence of an agent in the first sample, wherein the agent is electron dense microparticles comprising protein, for example, collagenase, such as, but not limited to, archaea collagenase (e.g., Archaemetzincin-1, Archeobacterial metalloproteinase-like protein 1, AMZ1); and detecting the presence of the agent in a second sample from the subject, wherein the second sample is not contacted with the test composition, wherein a higher concentration of agent in the first sample compared to the second sample indicates that the composition is suitable for use in treating the subject diagnosed with or at risk for developing heart disease or heart failure.

### 6 EXAMPLES

### Example 1

### Treatment of Aortic Atherosclerotic Plaques in Rabbits with Trans-Sialidase, PDTC, and Plant Extracts

The present study compares the effects of trans-sialidase (TS) enzyme derived from Trypanosoma cruzi, PDTC and one or more aged plant extracts derived from Allium sativum (AS), Ginkgo biloba (GB) and Zingiber officinale (ZO), on the reduction of aortic atherosclerotic plaques, lipid serum levels and infectious agent antigens at intima in rabbits receiving cholesterol-rich-diet.

### Material and Methods

White New Zealand male rabbits of approximately 2 months in age, weighing 2.2±0.5 kg were included in the study. The study lasted 12 weeks. The rabbits were divided into six different treatment groups. Group I animals received normal rabbit chow, while Groups II-VI received normal rabbit chow supplemented with 1% cholesterol. Animals received the diets for a period of 12 weeks. Groups III-VI also received anti-atherosclerotic treatment during the final 4 weeks of the study. The feeding and treatment schedule is shown in Table II.

TABLE II
Feeding and treatment schedule for the six study Groups.

| | Number of | | Anti-Atherosclerotic |
|---|---|---|---|
| | Rabbits | | Treatment (final 4 |
| Group | in Group | Diet | weeks of study) |
| GI. | 13 | Normal rabbit chow | None |
| GII. | 13 | Normal rabbit chow + | None |
| | | 1% cholesterol | |
| GIII. | 5 | Normal rabbit chow + | TS + PDTC |
| | | 1% cholesterol | |
| GIV. | 5 | Normal rabbit chow + | TS + PDTC + AS |
| | | 1% cholesterol | extract |
| GV. | 5 | Normal rabbit chow + | TS + PDTC + AS + |
| | | 1% cholesterol | GB extracts |
| GVI. | 5 | Normal rabbit chow + | TS + PDTC + AS + |
| | | 1% cholesterol | GB + ZO extracts |

Diet Preparation

Nuvilab® (Nuvital. Curitiba, PR. Brazil) was used as the normal rabbit chow in the study. Normal rabbit chow supplemented with 1% cholesterol was prepared by adding 10 g of cholesterol powder (Sigma—C 8503) dissolved in a solution of 50 ml ethylic ether and 100 ml 70% ethanol, to each Kg of normal rabbit chow

Trans-Sialidase (TS) Preparation

Trypanosoma cruzi were cultured according to Kloetzel et al. (Kloetzel et al., 1984, Trypanosoma cruzi interaction with macrophages: differences between tissue culture and bloodstream forms. Rev. Inst. Med. Trop. Sao Paulo., 26:179-85). Supernatant from the culture was filtered through a 1 μm pore filter in a vacuum chamber, or the supernatant was filtered through a 0.22 μm filter and concentrated by precipitation with 50% (NH₄)2SO₄. The precipitates were dialyzed against phosphate-buffered saline, and then passed through a tresyl-agarose column containing an immobilized anti-trans-sialidase monoclonal antibody. The column was washed with phosphate-buffered saline, followed by a 10 mM sodium phosphate, pH 6.5 wash. The trans-sialidase was eluted with a 3.5 mM MgCl₂, 10 mM sodium phosphate, pH 6.0 solution. The fractions eluted from the column were immediately filtered through a Sephadex G-25 column equilibrated with 20 mM Tris-HCl, pH 8.0, to remove MgCl₂. The trans-sialidase was further purified by passage through a Mono Q column equilibrated in 20 mM Tris-HCl, pH 8.0, and eluted with a linear gradient from 0 to 1 m NaCl in the same buffer.

The purified enzyme derived from the culture comprises a 400 kDa multimeric aggregate. The enzymatic activity of the purified trans-sialidase was measured according to methods described in International Patent Application No. PCT/BR01/00083, filed Jul. 3, 2001. Purified trans-sialidase used in the study had an enzymatic activity of 1.3 U/ml.

Plant Extract Preparation

Plant (Allium sativum (AS) cloves, Ginkgo biloba leaves (GB) and Zingiber officinale (ZO) raw) extracts were prepared by introducing sliced plant tissue into a 10-20% aqueous ethanol solution. The plant/ethanol mixture was adjusted to a final proportion of 40:60 plant weight:ethanol and stored for up to 12 months at room temperature in a dark, anaerobic environment (in a sealed bottle). Following storage, the plant mass/alcohol mixture was passed through Whatman qualitative filter paper grade 1, diameter 24 cm, pore size 11 μm. The liquid filtrate was then filtered again in a vacuum chamber with a 47 mm diameter glass microfiber filter, pore size 1.1 μm. Then filtrate was next filtered through successively smaller pores, in a tangential flow device (Minitan Ultrafiltration Millipore System—Millipore, Bedford, Mass., USA), using the microporous membrane packet (30,000 NMWL) that concentrates large particles. The filtrated portion of the extract was used in the study.

Trans-Sialidase (TS)+PDTC Anti-Atherosclerotic Treatment

Rabbits were treated with 0.25-0.5 ml/kg of a trans-sialidase+PDTC mixture injected intraperitoneally on alternative days. 1 ml of the treatment mixture comprised 0.8 ml of Trypanosoma cruzi culture supernatant (enzymatic activity of 1.3 U/ml) and 1.5 mg of PDTC (pyrrolidine dithiocarbamate ammonium salt from ICN Biomedicals Inc., Aurora, Ohio, USA.) dissolved in 0.2 ml of saline.

Trans-Sialidase (TS)+PDTC+Plant Extract Anti-Atherosclerotic Treatment

Animals were treated with the trans-sialidase+PDTC solution as described above along with 1 ml of a purified plant extract dilution containing an average of 1×10⁶ nanoparticles. The plant extract dilution was administered through intraperitoneal injections once per day during the four week treatment period. The purified plant extract dilution was generated by diluting an aged ethanolic plant extract 1:10 in water.

Serum Lipid Analysis

Serum lipid analysis was performed at the beginning and end of the 12 week experiment. To obtain the blood serum, a 10 ml blood sample was taken from each animal through cardiac puncture, and centrifuged at 1500 g for 15 min at 4° C. Total cholesterol, high-density lipoprotein (HDL) and triglycerides concentrations were determined by enzymatic methods (CHOD-PAP Merck®, USA. and GPO-PAP Cobas Mira, Roche).

Aortic Atherosclerotic Lesions Analysis

To analyze aortic atherosclerotic lesions, rabbits were euthanized with an intramuscular injection of 25 mg/kg Ketamine and 2-5 mg/kg Xilazina. Aorta were excised and opened longitudinally along the anterior wall, washed in saline, stretched on cardboard, and placed in 10% buffered formalin. Aorta were then stained with Sudan IV. Intimal positive areas stained in red by Sudan IV were measured by automatic detection using an image analysis system (Quantimet 500, Leica).

Histological examination of the aorta were also performed. A 1 cm thickness cross-section of the initial descending thoracic aorta were taken and embedded in paraffin. 5 μm serial sections of the cross-section were submitted to H&E stain and immunohistochemical detection of Mycoplasma pneumoniae (MP) and Chlamydia pneumoniae (CP) antigens, as previously described. (Fagundes R Q. Study of co-participation of natural infection by Chlamydophila pneumoniae and Mycoplasma pneumoniae in experimental atherogenesis in rabbits. Doctoral thesis presented at the Heart Institute of Clinical Hospital, in the Cardiology Sciences Post graduation Program of Sao Paulo University School of Medicine, Mar. 17, 2006). The percentage of area positive for infectious agent antigens on the immunostained slides was determined using an automatic color detection system (Image Analysis System Quantimet 500, Leica, Germany).

### Results

The mean and standard deviation values of percentage areas of fat plaques (macroscopically) and of MP and CP antigens at intima, and intimal area in 1 cm cross section are shown at table III. Lipid levels in the serum are reported at table IV.

### Atherosclerotic Plaques and Lipid Levels

The control group, Group I, which received normal rabbit chow and no ant-atherosclerotic treatment, did not develop plaques on the aortal walls. Trace amounts of MP and CP antigens on the aorta wall were detected, but in all cases, without development of atheroma plaques.

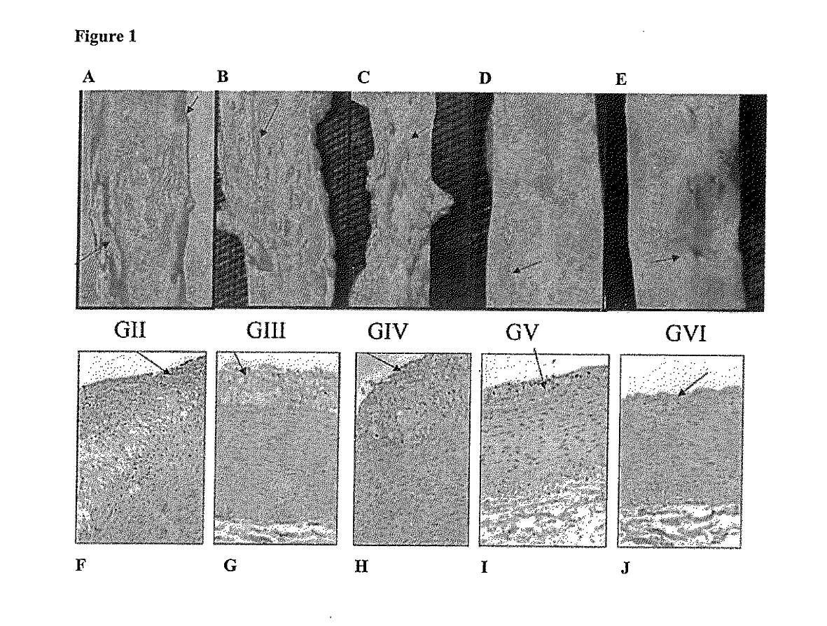

Group II, which received normal food supplemented with 1% cholesterol and no anti-atherosclerotic treatment, presented 75% coverage of the aorta intimal surface by severe lipid atheroma plaques stained with Sudan IV. (FIG. 1). The histology revealed that the plaques were comprised of 89% fat.

Group III, which received normal food supplemented with 1% cholesterol and treatment with TS+PDTC, exhibited 50% coverage of the aorta intimal surface by severe lipid atheroma plaques stained with Sudan IV. (FIG. 1).

Groups IV, V and VI, which received normal food supplemented with 1% cholesterol and treatment with TS+PDTC+Plant extracts, presented progressively smaller areas of atherosclerotic plaque coverage of the aorta wall (Table III). The addition of AS to the treatment regime reduced the levels of total cholesterol and HDL in the blood serum (Table IV), but did not reduce the % plaque area of atheroma (Table III), and induced a decrease in aorta perimeter, indicating a negative remodeling of the vessel. The addition of AS+GB to the treatment led to a significant reduction in both % area of intimal plaques and cholesterol levels in the serum. The most effective anti-atherosclerotic effect was observed with a complex of plant extracts from AS, GB and ZO, which reduced the area of the aorta wall covered by plaque to 11%, and returned lipid levels in the serum to normal levels (Table IV). Most of the remaining intimal plaques were fibrotic, largely free of foam cells (FIG. 1). Treatment with AS, GB and ZO extracts reduced both intimal area and % of intraplaque fat (Table III).

TABLE III
Intimal Area and Percentage Areas of Aorta Atheroma Plaques, Fat and Infectious Agents
in Aortic Plaques of 1% Cholesterol-Fed Rabbits Submitted to Different Treatments.

| | % area | % area | % Plaque area − | | Intima area |
|---|---|---|---|---|---|
| | C. pneumoniae+ | M. pneumoniae+ | macroscopic | % plaque fat | (mm²) |
| Group | Mean (SD) | Mean (SD) | Mean (SD) | Mean (SD) | Mean (SD) |
| GI | 0.007 (0.005) | 0.013 (0.012) | 0.0 (0.0) | 0.0 (0.0) | 0.0 (0.0) |
| GII | 23.50 (5.66) | 25.60 (3.96) | 75 (9) | 89 (5) | 75 (7) |
| GIII | 16.04 (0.60) | 12.81 (1.27) | 50 (3) | 50 (3) | 65 (4) |
| GIV | 12.60 (0.85) | 10.53 (0.51) | 67 (14) | 61 (10) | 61 (8) |
| GV | 8.60 (0.21) | 4.57 (0.51) | 42 (8) | 40 (14) | 39 (6) |
| GVI | 0.022 (0.005) | 0.02 (0.005) | 11 (1) | 17 (10) | 17 (2) |

TABLE IV
Cholesterol Fractions and Triglycerides Serum Levels of 1% Cholesterol-Fed
Rabbits Submitted to Different Treatments. Values shown are in mg/dl.

| | Total | | | | | | | |
|---|---|---|---|---|---|---|---|---|
| | cholesterol | | Triglycerides | | HDL | | LDL | |
| | Mean | | Mean | | Mean | | Mean | |
| Group | (SD) | p | (SD) | p | (SD) | p | (SD) | p |
| GI | 67 (31) | 0.17 | 64 (13) | 0.01 | 42 (7) | <0.01 | 33 (24) | 0.09 |
| GII | 1029 (237) | <0.01 | 115 (55) | <0.01 | 210 (52) | <0.01 | 775 (227) | <0.01 |
| GIII | 873 (82) | 0.09 | 95 (10) | 0.22 | 175 (17) | 0.08 | 743 (92) | 0.38 |
| GIV | 778 (58) | 0.02 | 86 (9) | 0.13 | 115 (11) | <0.01 | 635 (60) | 0.10 |
| GV | 408 (69) | <0.01 | 51 (6) | <0.01 | 90 (6) | <0.01 | 335 (29) | <0.01 |
| GVI | 53 (6) | <0.01 | 47 (8) | 0.26 | 36 (3) | <0.01 | 18 (6) | <0.01 |

GI—control group not receiving cholesterol diet;
GII—non treated;
GIII—received trans-sialidase (TS) and pyrrolidine dithiocarbamate (PDTC);
GIV—received TS + PDTC + Allium Sativum extract (AS);
GV—treated with TS + PDTC + AS + Ginkgo biloba extract (GB) and
GVI—received TS + PDTC + AS + GB + Zingiber officinale extract;
SD—standard deviation
p—represents difference regarding the respective values of the above group, except GI values which were compared with group GVI (level of significance 5%)

Mycoplasma pneumoniae and Chlamydia pneumoniae Antigens

Treatment with TS+PDTC (Group III) reduced the percent area of MP antigen expression from 25.6±3.96 to 12.81±1.27 (p<0.01) and CP antigen expression from 23.50±5.66 to 16.04±0.60 (p<0.001) as compared to Group 11 animals that received no anti-atherosclerotic treatment. Addition of plant extracts caused a progressively more significant decrease in percentage area positive for CP and MP antigens at intima. When all three plant extracts were used in the anti-atherosclerotic treatment, the reduction was more effective. Use of all three extracts reduced the percentage of total area expressing MP antigen to 0.02±0.005 and CP antigens to 0.022±0.005. These values were similar to the control group (Group I) (Table III). Macroscopic and microscopic aspects of different groups are exemplified at the FIG. 1.

### Conclusion

In conclusion, the present study indicates a new formulation for the treatment of atherosclerosis, using a combination of T. cruzi trans-sialidase, PDTC and three aged plant extracts: Allium sativum, Ginkgo biloba and Zingiber officinale. Treatment with these compounds was effective in reducing intimal accumulation of both fat and C. pneumoniae plus M. pneumoniae antigens. The lipid serum levels returned to normal levels even in the permanence of a cholesterol rich diet.

### Example 2

### Treatment of Human Patients Exhibiting High Total Cholesterol and Ldl Levels with Trans-Sialidase and Plant Extracts

Three volunteers who presented high levels of total cholesterol and LDL cholesterol fraction in blood serum were treated with purified trans-sialidase and aged plant extracts.

### Materials and Methods

Plant Extract Preparation

Plant (Allium sativum (AS) cloves, Ginkgo biloba leaves (GB). Zingiber officinale raws (ZO) and Pfaffia paniculata (Brazilian ginseng) roofs (GS)) extracts were prepared by introducing sliced plant tissue into a 10-20% aqueous ethanol solution. The plant/ethanol mixture was adjusted to a final proportion of 40:60 plant weight:ehtanol and stored for up to 12 months at room temperature in a dark, anaerobic environment (in a sealed bottle). Following storage, the plant mass/alcohol mixture was passed through Whatman qualitative filter paper grade 1, diameter 24 cm, pore size 11 μm. The liquid filtrate was then filtered again in a vacuum chamber with a 47 mm diameter glass microfiber filter, pore size 1.1 μm. Then filtrate was next filtered through successively smaller pores, in a tangential flow device (Minitan Ultrafiltration Millipore System, Millipore, Bedford, Mass., USA), using the microporous membrane packet (30,000 NMWL) that concentrates large particles. The filtrated portion of the extract was used in the experiments.

Recombinant Trans-Sialidase Purification

Recombinant trans-sialidase was produced and purified from the Escherichia coli strain BLB21 DE3 inserted with a pTSII plasmidium comprising the T. cruzi trans-sialidase gene as described in International Patent Publication WO/2002/002050 by Higuchi et al., published Jan. 10, 2002.

The protein concentration of 5 mg/ml was produced as measured with a spectrophotometer. The recombinant trans-sialidase was diluted in a buffer liquid (TBS+BSA 0.2%), and the activity was measured according to previously described methods (International Patent Publication WO/2002/002050). The purified enzyme was diluted 1:10,000 and 1:100,000 resulting in enzymatic activities of 15,000 and 5,000 CPM, respectively. For human oral administration, the trans-sialidase was diluted 1:1,000,000 (0.005 mg/ml) in MilliQ purified water, and stored at 4° C.

Preparation and Administration of Oral Drug

Equal proportions of pure extracts from Allium sativum (AS); Ginkgo biloba (GB) Zingiber officinale (ZO) and ginseng (GS) were mixed. The mixture was then diluted 1:1 in thermal water (from Irai, RS, Brazil), which was previously boiled and filtered.

Trans-sialidase diluted 1:1,000,000 (0.005 mg/ml) was administered to the subjects. A mean of 200 ul to 500 ul (4-10 drops) of diluted trans-sialidase was added in a glass of water and ingested daily.

Three volunteers who presented high levels of total cholesterol and serum LDL cholesterol were treated with the oral compositions for a minimum of 30 days to over one year. The volunteers were administered orally 200 ul of the diluted plant extract composition 2×/day, and 200 ul of the diluted trans-sialidase composition 1×/day. The patients were also being treated with other anti-cholesterol drugs (statins). Following treatment, the volunteers presented normal total cholesterol and serum LDL cholesterol levels, wherein the mean level of decrease in serum cholesterol levels following treatment was 20%. This decrease was observed even if statins had been previously used to lower serum cholesterol levels.

### Example 3

### Tobacco Extracts Contain Large Lipidic Pathogenic Archaea that can be Removed NY Incubation in Thermal Water

### Tobacco Extracts:

Tobacco extracts were obtained by removing the contents from a packet of commercial tobacco cigarettes, and adding the contents to 80 ml of water. The tobacco/water mixture was then mixed with 500 ml of ethanol (92% ethanol). The tobacco/water/alcohol mixture was then aged in a sealed bottle for 12 months. Following 12 moths of aging, the mixture was filtered filtered through Whatman qualitative filter paper (grade 1, diameter 24 cm, pore size 11 μm). The filtrate was then filtered a second time through vacuum chambers comprising a 47 mm diameter glass microfiber filter with a pore size of 1.1 μm.

The filtrate was analyzed with fluorescent and electron microscopy as described in U.S. Patent Application Publication No. 20050142116. Fluorescent microscopy of filtrate stained with acridine orange showed a large number of both large particles and nanoparticles containing DNA or RNA, but the filtrate was predominated by the large particles.

Analysis of the filtrate with electron microscopy showed that the two particles were the two types of archaea described previously: very small and clear structures of about 0.03-0.15 μm in diameter (see FIG. 4), which correspond to non-pathogenic archaea; and large particles (0.15-0.24 μm), along with other electron dense lipidic structures, which correspond to pathogenic archaea (see FIG. 5). The large archaea particles may also be observed as round brilliant red particles under fluorescent microscopy.

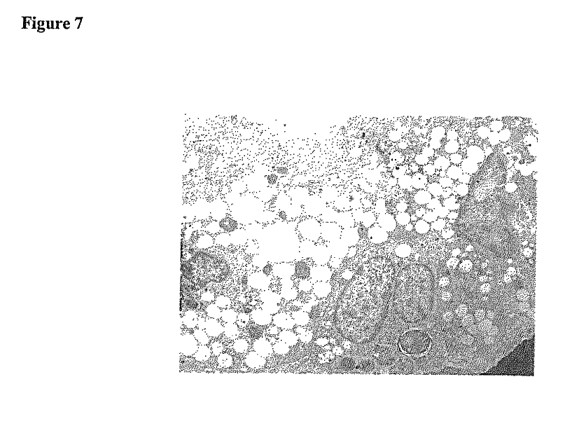

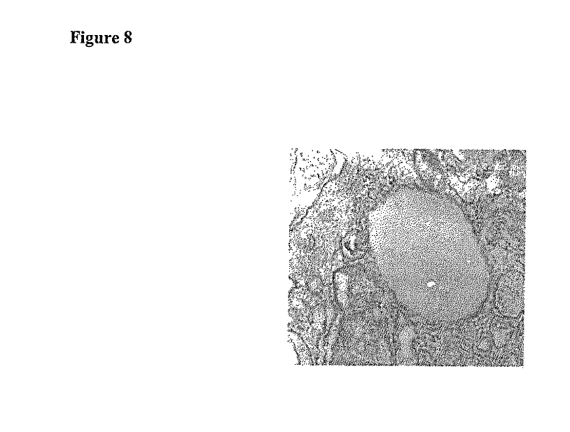

The pathogenic large particle archaea are also found in human periadventitial adipose tissue of atherosclerotic aortic aneurysms Analysis of human periadventitial adipose tissue of atherosclerotic aortic aneurysms with electron microscopy showed that this tissue contained a large number of the large lipidic particles surrounded by inflammatory infiltrate archaea surrounded by inflammatory lymphocytes (FIG. 6) suggesting that the particles are recognized as foreign structures by the immune system. High magnification of theses lipidic particles (FIG. 7) shows that the particles contain a clear external membrane, indicating that these particles correspond to microbes (large lipidic archaea), and not to lipidic droplets in the cytoplasm. These lipidic large archaea have the same morphology as the large particles that predominate tobacco extract, and as shown in FIGS. 5 and 6.

Preparation of the Therapeutic Extract from Tobacco:

As described previously, diluting and aging plant ethanolic extracts results in an extract enriched with non-pathogenic archaeas (see U.S. Patent Application Publication No. 20050142116). For example, diluting an ethanolic plant extract with thermal medicinal water (from Irai city in South of Brazil) in a proportion of 1:10 (extract/water), and aging the mixture for 30 days, results in a reduction of the large lipidic archaea particles, while retaining the small non-pathogenic archaea. Extracts with enriched non-pathogenic archaea have been shown to be useful in the treatment of atherosclerosis and lowering serum lipids. Accordingly, tobacco extract prepared as described above and aged for 12 months was diluted 1:10 in thermal water, and aged for an additional 30 days.

Atherosclerosis was induced in a rabbit by feeding the rabbit with a high cholesterol diet (5% cholesterol) for 12 weeks. Following the 8 weeks of the feeding period, 0.5 ml samples of the aged 1:10 tobacco extract/thermal water mixture (which was enriched with non-pathogenic archaea) was subcutaneously injected into the rabbit's ear, twice a week, during the last 4 weeks of cholesterol enriched diet program. The animal was then sacrificed followed by macroscopic and microscopic analysis of the ascending and descending thoracic aorta. Both analyses did not show any atheroma plaques in the ascending or descending thoracic aorta, which are normally present following a cholesterol enriched feeding program (see FIG. 1).

### Conclusion

The use of thermal medicinal water to dilute aged ethanolic plant extracts is effective in eliminating undesirable pathogenic large particle archaea, and preserving non-pathogenic archaea present in the extracts. Such an observation is observable by direct visualization of the plant extract mixture with fluorescent microscopy before and after diluting the extract with thermal water. Thus, the use of thermal water to purify plant extracts may increase the therapeutic and medicinal properties of the extracts. For example, non-pathogenic archaea present in tobacco extract may be enriched through purification with thermal water, and used to treat cholesterol induced atherosclerosis. One hypothetical mechanism of the success of such a treatment is that in human atherosclerotic lesions, such as aneurysms or unstable plaques that cause myocardial infarction, there are higher numbers of pathogenic archaea. These pathogenic archaea in the lesions may be increased by the use of tobacco products. Surprisingly, increasing the non-pathogenic archaea present in tobacco extracts by diluting the extracts with thermal water, may enable tobacco to be used as a treatment to combat the pathogenic archaea and atherosclerosis.

### Example 4

### Evaluation of Serum Sample from Patient with Coronary Artery Disease (CAD)

Lipoprotein from Mycoplasma pneumoniae and lipopolysaccharide (LPS) from Chlamydia pneumoniae were detected in the serum, by immunoelectron microscopy.

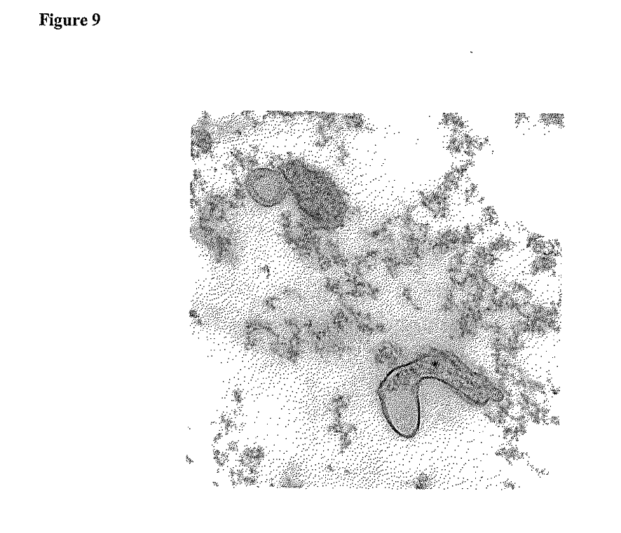

One ml of serum from the patient was processed to detect lipid particles, such as archaea. Lipoprotein from Mycoplasma pneumoniae was in greater amount in the serum of atherosclerotic patients and LPS from Chlamydia pneumoniae was increased in the serum of patients with acute myocardial infarction. Particles of C reactive protein (CRP) were also detectable by immunoelectron microscopy and their number increased in correlation with the number of mycoplasmal lipoprotein. The mean numbers of CRP and Mycoplasma pneumoniae particles/mm² of electron microscopy photos were higher in CAD patients (1.45+/−0.50 and 1.32+/−1.35) than in healthy subjects with similar age (1.05+/−0.29 and 0.26+/−0.20) (p<0.01), and were correlated with each other only in atherosclerotic patients group. The success of treatment may therefore be determined by measuring the particles of lipoprotein from mycoplasma, LPS from Chlamydia pneumoniae or pathogenic archaea in the serum, as shown in FIG. 9.

### Example 5

### Detection of Archaea and Microbes in Serum

Archaea and other microbes were concentrated from human serum and plasma using a modified technique for separating mitochondria and other organelles from tissue (Bustamante et al., Biochemical and Biophysical Research Communications. 334:907-910, 2005).

To separate serum from blood, 10 ml of blood was incubated for 1 hour in a water bath to form a blood clot that could be separated from the supernatant. Serum was isolated from the supernatant and blood clot by centrifuging the mixture at 2000 rpm for 5 minutes.

1 ml of serum was then prepared for electron microscopy by adding 1.0 ml of glutaraldehyde fixative and 1.0 ml of Osmium tetroxide to the serum. The treated serum was then centrifuged, and the resulting pellet was processed and embedded in glutaraldehyde. Thin sections of the pellet were prepared and examined under an electron microscope.

H medium (200 mM D-mannitol, 70 mM sucrose, 2 mM Hepes and 0.5 g/L defatted BSA, pH 7.2) was added to the remaining serum and centrifuged at 11400 rpm for 12 minutes at 4° C. The resulting supernatant and pellet were collected. The pellet was resuspended in H medium, and both the pellet and supernatant were analyzed using electron microscopy and PCR.

### Results

The supernatants of the serum were predominantly rich in clear double membrane surrounding vesicles containing archaeal DNA, while the pellets contained mainly electron dense structures suggestive of ricketsia, Chlamydia, spirochete, and mycoplasma bodies.

### Example 6

### Chagas Cardiopathy is Associated with Archaea

Archaea-like bodies were found in association with Chagas' disease in two morphological types: one with electron dense lipidic content (EDL) and other with electron lucent content (ELC) (Higuchi et al. Mem Inst Oswaldo Cruz 2009; 104 (Suppl I): 199-207). T. cruzi is the aetiological agent of Chagas' disease, but most of the patient become asymptomatic during all their lives, only around 30% of the infected patients develop Dilated Cardiomyopathy related to an apparently autoimmune myocarditis. Proteasomes are organdies that rid the cells of abnormal proteins and trypanosoma was discovered to carry proteasomes from archaea, and it has been considered an evolutionary endosymbiotic mechanism.

Archaeal-like organdies and archaeal genome were present in all endomyocardial samples. Electron microscopy electron dense lipidic (EDL) organelles containing archaeal DNA in chagasic endomyocardial biopsies (EB) were observed. There was a significant negative correlation between numbers of ED organelles vs. amount of archaeal DNA dots outside of ED in IF (indeterminate asymptomatic form) (r=−0.46), and lack of correlation in HF (heart failure) group (r=−0.11), suggesting that EDL archaea-like organelles are associated with IF patients. Patients with chagasic cardiomyopathy had smaller archaeal organelles (0.10±0.08 vs. 0.24±0.12 μm, p<0.05) and lower number of archaeal genome dots (0.07±0.07 vs. 0.28±0.10/μm², p<0.05), as compared to patients without chagasic cardiomyopathy. In patients without heart disease there was a negative correlation between numbers of archaeal organelle bodies and the number of archaeal genome dots in the extracellular matrix (r=−0.77); in patients with heart disease there was a positive correlation (r=0.69).

The control normal case did not show intramyocyte archaeal-like organelles. Scarce archaeal DNA points were seen in foci of extracellular matrix (mean of 0.41 μm²).

Thus, archaeal DNA and different types of archaeal-like bodies are present in chagasic patients that may be related with prevention of “autoimmune” myocarditis in indeterminate form or inducing inflammation and fibrosis in heart failure CC (chronic cardiopathy) form.

### Example 7

### Archaea and Microbes May be Detected in the Serum of Chagas Disease Patients

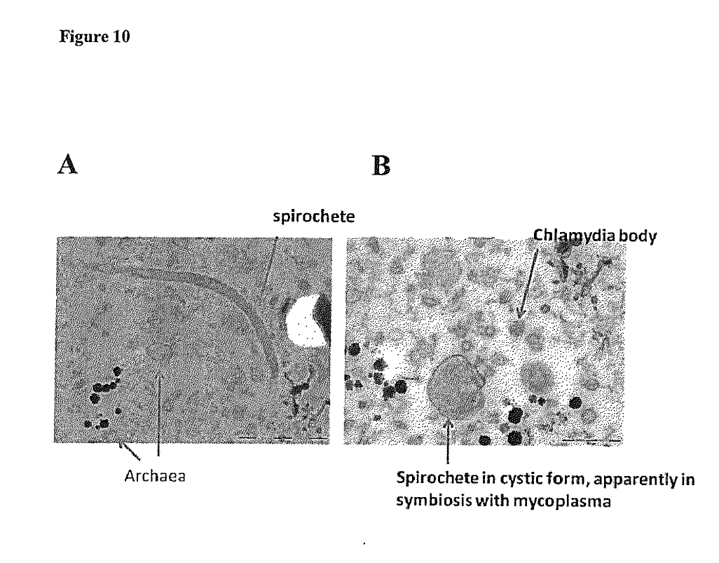

The serum of 3 patients with dilated chagasic cardiomyopathy were analyzed. A large amount of different primitive microbial bodies associated with empty pathogenic archaea were observed, as demonstrated in FIG. 10. This amount of microbial bodies was not seen in chronic atherosclerotic patients, nor in healthy individuals. This finding strongly suggests that the analysis of the serum may be an important biomarker for specific therapeutic proposals.

### Example 8

### Histology of the Myocardium from “Normal”Donor Hearts Vs. Dysfunction of the Organ after Heart Transplantation

Donor heart myocardial histology were evaluated, focusing on inflammation which may correlate with mortality after heart transplantation (HT) within 30 days after HT.

Methods—20 donor hearts were biopsied at the right ventricle immediately before the HT surgery and analyzed histologically and by immunohistochemistry (T cells and CD68 macrophages). The myocardial histology of patients who died due to dysfunction of the transplanted organ was compared with the others.

Results—Patients with heart dysfunction had a significant higher amount of lymphocytic infiltrate in the myocardium previously to the HT (12+/−4.6 cells/mm²) compared with those who did not die (3.75+/−3.57 cells/mm²) (p<0.011).

In conclusion, the inflammatory infiltrate in the myocardium may be a risk biomarker for early mortality after HT.

Additionally, studies at the electron microscopy level in an ongoing study suggests the presence of pathogenic archaea in the serum and in the myocardium of the donor hearts who died in the period of 30 days after HT.

### Example 9

### The Associate of Archaea, Mycoplasma and Chlamydia with Cancer Cells

Intimal association of three different microorganisms in human cancer cells have been observed in morphological studies. The three microorganisms were mycoplasmas, chlamydias and archaeas.

Chlamydia pneumoniae, Mycoplasma pneumoniae, Mycoplasma pulmonis and archaeas were detected in 22 of 23 different malignant neoplasias using immunohistochemistry, in situ hybridization, confocal laser microscopy, immunofluorescence and electron microscopy techniques. Similarly, analysis of primary cell cultures of colon adenocarcinoma cells using immunofluorescence, electron microscopy and PCR showed that the microorganisms were also present. Except for one renal clear cell carcinoma without nuclear aberration, all of the neoplasias were highly positive for mycoplasmal antigens. In situ hybridization revealed Mycoplasma pneumoniae DNA in the cytoplasm and nuclei of most of the malignant cells. Additionally, the Mycoplasma pneumoniae DNA was also observed to be present in inflammatory cells adjacent to the malignant cells.

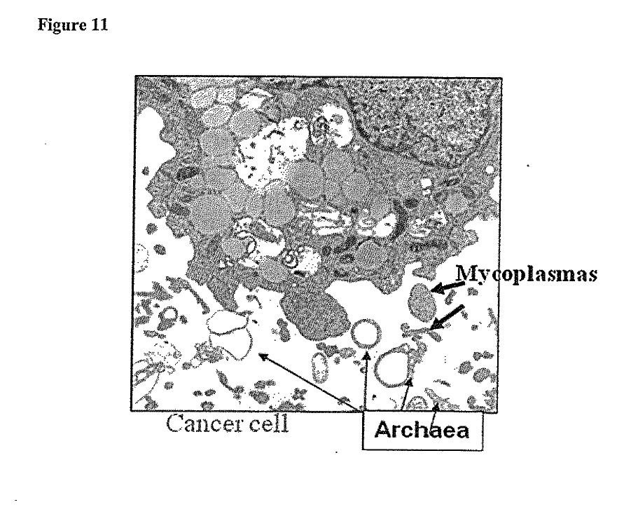

Electron microscopy showed filiform prolongings on the neoplastic cell surface and rounded tubule structures. Such structures are morphologically characteristic of mycoplasmas and archaeas (e.g., the presentation of double envoltory membrane, rounded electron dense vesicles resembling lysosome, and other irregularly shaped vesicles with electron lucent content, see FIG. 11). Furthermore, C. pneumoniae elementary bodies were detected in the cytoplasm of the neoplastic cells.

As such, malignant pleomorphic cells contain different species of mycoplasma, Chlamydia pneumoniae and archaeas. The intimal association of these agents may result in an increased virulence of these agents, promoting cellular invasion. Accordingly, detection of such agents in tissue sample from a subject can be used as a means for diagnosing cancerous cells, or for monitoring the success of a cancer treatment.

### Example 10

### Archaea as a Biomarker of Cardiotoxicity in Cancer

Many anti-cancer agents may have significant potential for cardiovascular toxicities that include QT prolongation and arrhythmias, the induction of myocardial ischemia and infarction (e.g., resulting from treatment with antimetabolites), hypertension or venous and arterial thrombo-embolism (e.g., resulting from treatment with the anti-angiogenic agents bevacizumab, sorafenib, sunitinib, and pazopanib), and cardiac dysfunction or heart failure. The latter is variable in severity, may be reversible or irreversible, can occur immediately or as a delayed consequence of treatment, and may involve diastolic as well as systolic dysfunction. Biomarkers that precociously indicate the potential of cardiotoxic side effects of cancer treatment would be beneficial (see, Eschenhagen et al., “Cardiovascular side effects of cancer therapies: a position statement from the Heart Failure Association of the European Society of Cardiology.” European Journal of Heart Failure, 13:1-10, 2011).

The presence of archaea in the serum of cancer patients, as well as the presence of pathogenic electron lucent archaea, similar to those described above in chagasic patients, in cancer patient serum, are associated with cancer treatment cardiotoxicity, The detection of these biomarkers can be used to diagnose the presence or likelihood of developing cardiotoxicity in cancer patients receiving cancer treatment.

The sera of 5 cancer patients was examined using the same technique as described above in the analysis of the sera of atherosclerotic patients. Fluorescent Qdots were used to observe numerous brilliant rounded microvesicle at 20× magnification. Analysis of these structures using electron microscopy showed the structures to be electron dense archaea-like bodies. The morphology of the structures was similar to those seen in the cancer biopsies described above in Example 9. One of 5 patients studied exhibited heart failure due to cardiotoxicity related to cancer treatment. This patient's serum presented many electron lucent archaea. Accordingly, electron lucent archaea, as seen in chagasic cardiopathy, may be important for the pathogenesis of cardiotoxicity heart failure in cancer patients and may be used as a biomarker for diagnosing the presence, or risk of developing cardiotoxicity.

### Example 11

### In Vitro Treatment of Cancer Cells and Serum from Heart Failure Patients Using Transialidase and Nanoparticles Purified from Plant Extracts

An in vitro assay was developed for identifying compositions effective for treating a subject diagnosed with or at risk for developing a disease associated with undesirable cell proliferation and fibrosis, such as atherosclerosis, high total serum cholesterol, serum LDL, serum HDL or triglyceride levels, cancer or cardiotoxicity due to cancer treatment, heart disease, for example, cardiomyopathies resulting from Chagas disease.

The in vitro assay utilizes cell cultures of a sample derived from a patient, for example, a cancer cell sample. The cell culture is then contacted with a test composition to determine if the test composition reduces the amount of microbes, microbe-like particles or microbe nucleic acid present in the culture following cell lysis, for example, following apoptosis, compared to cells lysed that are not contacted with the test composition. The assay can also be used to select compositions that both lyse the cells and reduce the amount of microbes, microbe-like particles or microbe nucleic acid present in the culture following cell lysis.

The in vitro assay can also utilize a serum, blood or plasma sample from a subject, wherein a test composition is contacted to the sample to determine if the test composition reduces the amount of microbes, microbe-like particles or microbe nucleic acid present in the sample compared to a sample not contacted with the test composition.

The in vitro assay is useful for determining what combinations and concentrations of protein capable of removing sialic acid residues, metal chelator and one or more purified plant extracts comprising therapeutic nanoparticles are most effective for inducing a therapeutic response in a subject.

### Treatment of Cancer Cell Culture

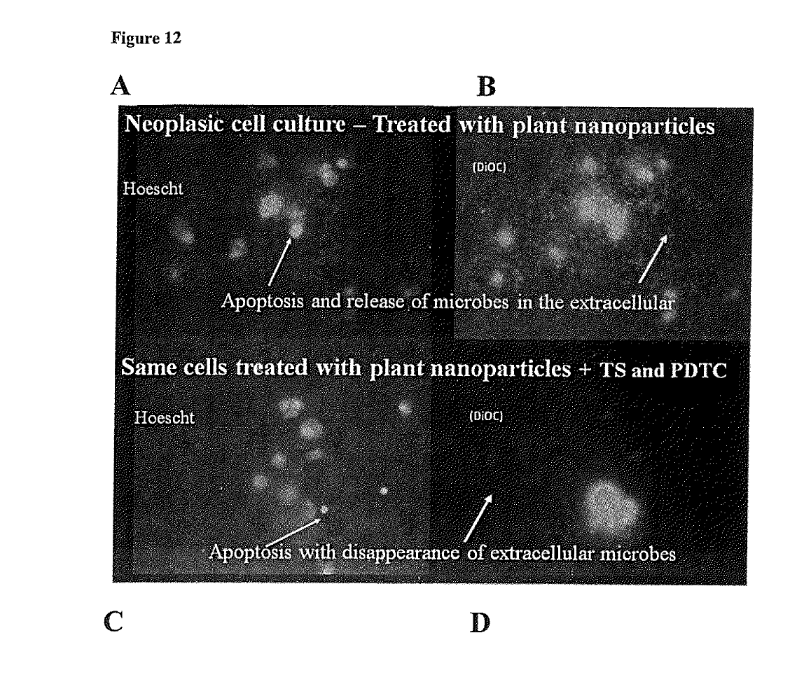

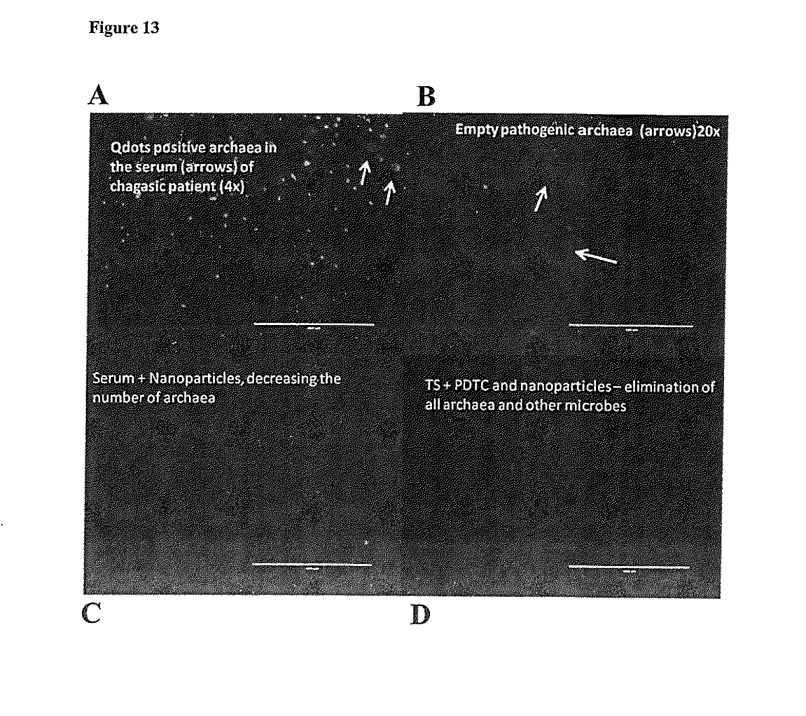

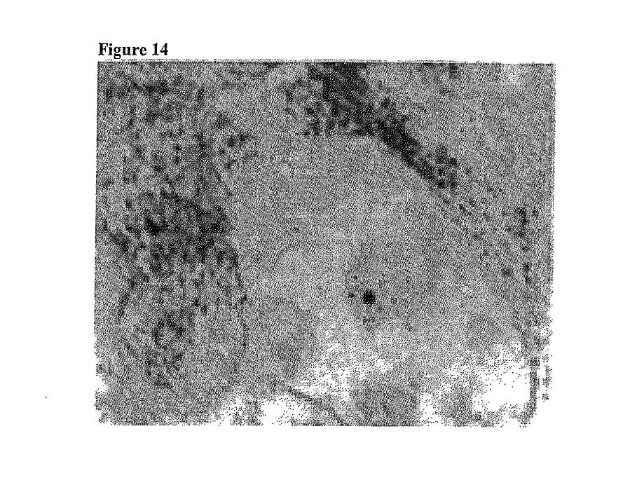

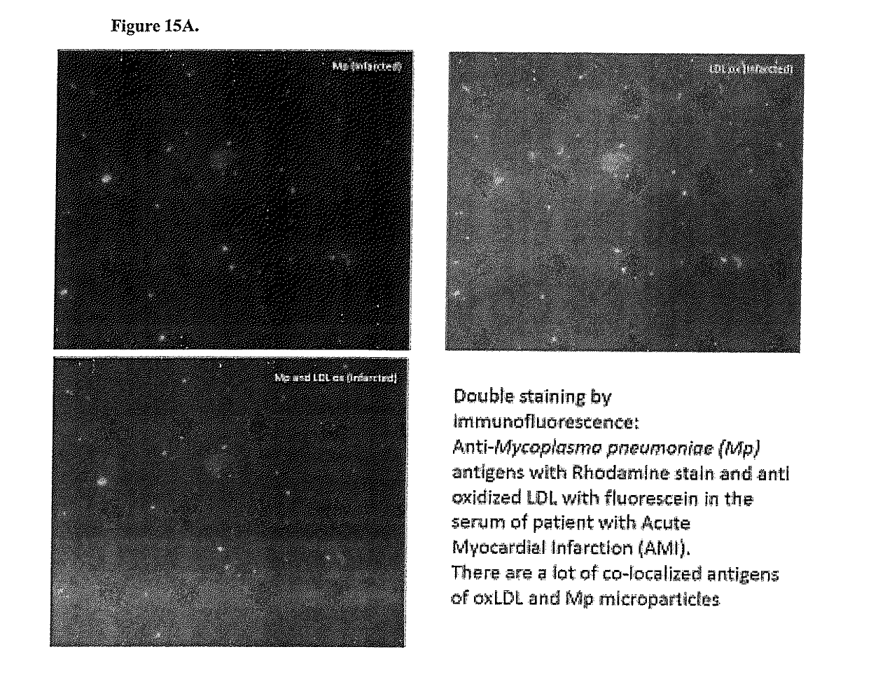

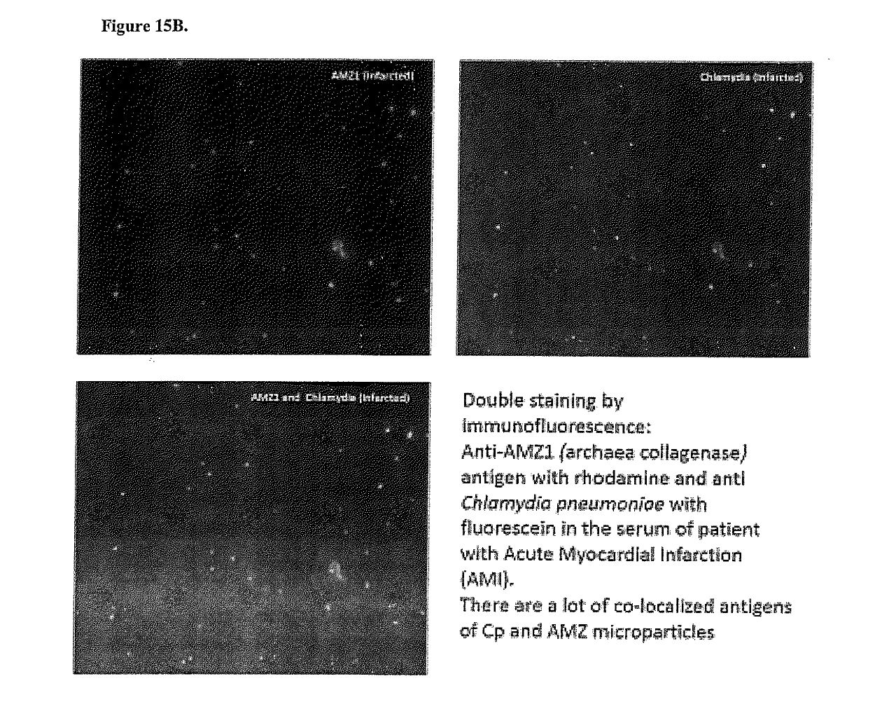

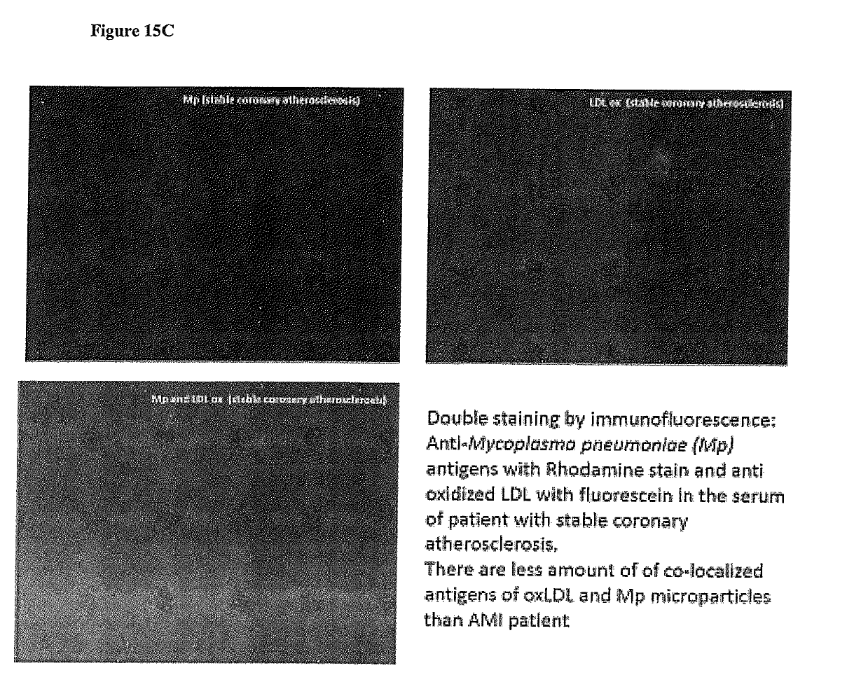

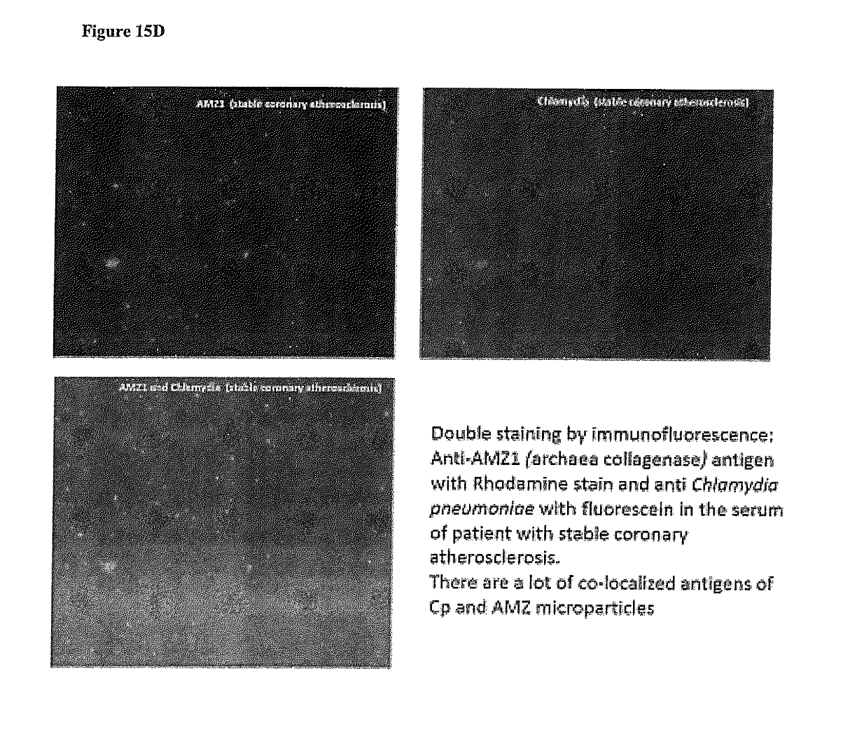

Slides containing 8 wells were prepared with primary cultures of adenocarcinoma cells (containing 5×10⁴ cells in 2.2 ml of culture medium). 50 ul of nanoparticles (a mixture of nanoparticles prepared from Gingko biloba, Zingiber officinalis, Golden root (Scutelaria baicalensis) and Dendrobium moschatum, prepared as previously described), were added to each well. Addition of the nanoparticles resulted in apoptosis of the neoplastic cells and release of microbes into the extracellular medium. The microbes were detected with the use of DIOC, which stains mitochondria (see FIGS. 12A and B).

In a second set of wells, 50 ul of the nanoparticles were added along with 45 ul of a solution comprising trans-sialidase (TS) (prepared as described previously with TS 1:1000+2 mg/ml PDTC). The composition comprising the nanoparticles and TS solution resulted in a higher level of apoptosis compared to use of nanoparticles alone, and further, the composition reduced the amount of microbes present in the extracellular medium following apoptosis (see FIGS. 12C and D).

Treatment of Serum Archaea from Patients with Heart Failure

Plant extract nanoparticle combinations were tested in the in vitro assay system for the ability of the combinations to reduce archaea present in serum samples from chagasic patients and patients with cardiotoxicity due to cancer treatment.

The effect of nanoparticles on sera from chagastic patients and treated cancer patients with cardiotoxicity was examined according to the following procedure: 1 ul of Qdot (Invitrogen) and 3 ul of nanoparticles were added to 20 ul of serum in individual wells of an 8 well slide. Each sample was observed using undirected fluorescent microscopy.

The composition most effective in reducing the amount of microbes present in the sera samples was a composition comprising nanoparticles derived from the extract of the following plants: Ginkgo biloba, Zingiber officinalis, Golden root (Scutelaria baicalensis) and orchid (Dendrobium moschatum) in combination with 3 ul of trans-sialidase diluted 1,000,000 fold in association with PDTC (see Example 13A-D).

The in vitro assay system described herein can therefore be used to detect the presence of microbes, for example, archaea, microbe-like particles, and/or microbe nucleic acid in a serum, blood or plasma sample, or in cell culture, and to assay compositions comprising nanoparticles prepared from plant extracts, trans-sialidase and PDTC for their effectiveness in reducing the presence of such microbes, microbe-like particles, and/or microbe nucleic acid in the samples. Compositions effective in reducing the amount of microbes, microbe-like particles, and/or microbe nucleic acid in the samples are selected for use as therapeutic agents for the treatment of a disease associated with undesirable cell proliferation and fibrosis, such as atherosclerosis, high total serum cholesterol, serum. LDL, serum HDL or triglyceride levels, and heart disease or heart failure. Heart disease or heart failure may be caused by injury, for example, by Chagas disease, dilated cardiomyopathy or cardiotoxicity during cancer treatment.

### Example 12

### Detection of Microparticles Comprising Archaeal DNA in Heart and Serum Samples from Chagasic Patients with the Indeterminate Form of Chagas Disease or Chagasic Cardiomyopathy

Introduction

Microparticles (MP), small vesicles released from the plasma membrane of activated or apoptotic cells, are progressively receiving larger attention as both potential biomarkers, and as a tool to evaluate tissue injury. MPs are abundantly released in patients with cardiovascular diseases and heart failure, tumors, neurological diseases, etc., and the serum MPs might be diagnostic or prognostic.ⁱ Circulating MPs from septic shock patients injected in mice caused increased expression of extracellular superoxide dismutase in the heart, suggesting that these MPs might participate in heart failure development.ⁱⁱ On the other hand, immunoadsorption (IA) treatment to remove cardiotoxic autoantibodies in patients with chronic inflammatory dilative cardiomyopathy (iDCM) improved endothelial and myocardial function in association with a significant drop in circulating MPs.ⁱⁱⁱ

MPs are believed to bear phospholipids and membrane proteins of their mother cell (e.g. CD144, CD146 and CD31 for ECs, CD42 and CD61 for platelets and CD45 for leucocytes) allowing their differentiation. In atherosclerosis, MPs derived from endothelial and inflammatory cells as well as platelets are increased, and is suggested that endothelial dysfunction may be an independent predictor of adverse events that could be assessed quantitatively by measurement of CD31+/annexin V+MP plasma levels, adding information about cardiovascular outcomes.^{iv} The quantification of MPs however, through characterization by size, antigen or RNA content does not distinguish clearly different clinical conditions^{v}. The ultrastructural morphology of MPs has been poorly explored. Electron microscopy evaluation is a promissory tool for MPs analysis.

Heart failure occurs in around 30% of chagasic patients (CC), most of the individuals infected by the parasite Trypanosoma cruzi (T. cruzi) remaining in asymptomatic clinical condition during all life called indeterminate form (IF). Studies with endomyocardial biopsies in the 1980's showed that CC patients present fibrosing lymphocytic myocarditis^{vi}, with lymphocytes injuring non infected myocytes^{vii}, and with unspecific ultrastructure alterations^{viii}. Antigens^{ix}, ^{x}, ^{xi}, DNA^{xii} from T. cruzi were found in the inflammatory infiltrate of the myocardium from CC patients, but in scarce quantity to explain the intensity of inflammatory infiltrate, with unbalanced immune response^{xiii}, ^{xiv}, suggesting that other factors beyond the parasite are present^{xv}, ^{xvi}.

It was hypothesized that MPs may have a role in the development of myocarditis and heart failure (HF) in Chagas' disease. In the present study, endomyocardial biopsy samples from previous studies were examined using electron microscopy. Two types of MPs were seen in the myocardium of chagasic patients: an electron dense (ED) with lipidic content, predominantly in the indeterminate form (IF) group and one with an electron lucent (EL) content, predominantly found in CC form. Both microparticles have double external membrane, a characteristic of microbes, such as the primitive archaea^{xvii}.

Archaea is a highly diverse and abundant group of prokaryotes, and include a number of “extremophiles” that thrive in such environments as hot springs, salt lakes, and submarine volcanic habitats^{sviii}. Recent molecular studies have revealed that archaea are commonly mesophilic^{xix}, and could be living in mammals or parasites. Archaeal-like genes were described in persistent pathogens apparently conferring metabolic capabilities as adaptation strategies for survival even in hostile host niches^{xx}. The possibility that Trypanosoma cruzi may carry archaeal genes is showed by the presence of an archaeal like M32 metallocarboxipeptidase and proteasomos consistent with a common ancestor archaeal-eubacterial in T. cruzi^{xxi},^{xxii}. M32 metallocarboxipeptidase is important for degradation of myocardial cell proteins during intracellular trypanosome growth; archaeal proteasomes is capable to degrade aggregation-prone proteins, reducing their cellular toxicities in mammalian cells^{xxiii}, and decreasing myocardial inflammation. Proteasomes are organelles that rid the cells of abnormal proteins and trypanosoma was discovered to carry proteasomes from archaea (archaeosomes) probably by evolutionary endosymbiotic mechanism. We observed by electron microscopy electron dense lipidic (ED) MPs containing archaeal DNA in chagasic endomyocardial biopsies (EB) with nanovesicles reminding proteasomes, which might be riding abnormal proteins in IF chagasic patients.

In the present work we investigated through electron microscopy if MPs present in endomyocardial biopsies and/or serum from chagasic patients, in respect of ultrastructural morphologies (archaeal DNA content, electron dense or electron lucent aspect and size) could differentiate two clinical forms: with heart failure chronic cardiac (CC) disease versus asymptomatic indeterminate form (IF).

### Material and Methods:

Endomyocardial biopsies from 6 CC and 5 IF chagasic patients and 5 fragments from normal heart (NH) donors were studied by in situ hybridization with electron microscopy. A generic probe was used for detecting biotinylated archaea DNA (ARCH 915:

(SEQ ID NO: 5)
GTGCTCCCCCGCCAATTCCT.

The grids were incubated in sodium metaperiodate solution 0.5 M for 15 minutes at room temperature (RT) for removing araldite resin and expose the antigenic sites. Then the sections were permeabilized with proteinase K solution 1:10,000 for 2 minutes, followed by denaturation with 0.5 N NaOH solution for 2 minutes and blocking of nonspecific proteins for 30 minutes. Then the grids were incubated with probe ARCH 915 40 ng/μl for 20 hours at 45° C. in a moist chamber.

After incubation, the grids were rinsed in stringency solution for 3 minutes at 50° C. and incubated with streptavidin labeled with colloidal gold 10 nm for 1 hour at RT. Then they were counterstained with uranyl acetate and 5% lead citrate and examined in a transmission electron microscope.

Mean numbers/electron micrography of ED and EL organelles and DNA positive dots in and outside of the organelles were counted analyzing 10 electron micrographies with 17.500× magnification of each case. The mean diameter of ED MPs and EL MPs, their amount and the content in archaeal DNA was counted. A comparison was performed by student T test

### Isolation of MPs in Serum of Chagasic Patients

Samples of 10 ml of whole blood from 2 groups were studied: IF (8 patients) and HF (7 patients). Serum was obtained by centrifugation for 5 minutes at 250 g.

The MPs were isolated according to the procedure described in Bustamante et. al.^{xxiv} with some modifications.

Serum (0.4 ml) was incubated with buffer containing 200 mM D-mannitol, sucrose 70 mm, 2 mM Hepes and 0.5 g/L BSA pH 7.2 for 1 hour at room temperature. The samples were centrifuged for 12 minutes x9500 g. The supernatant and pellet were collected for transmission electron microscopy analysis.

### Rapid Inclusion Method for Transmission Electron Microscopy

The inclusion was made following the procedure described by Duarte et. al,^{xxv} with some modifications.

After separation of fractions, the samples were fixed in 3% glutaraldehyde at 4° C. for 3 hours, post-fixed solution in 1% osmium tetroxide at 4° C. for 2 hours, and centrifuged for 5 minutes to pellet formation. Samples were then washed in saline solution and incubated in 0.5% uranyl acetate for 3 hours at 4° C. The pellets were dehydrated in 70% ethanol and 2,2-dimethoxypropane acidified, followed by fixation in acetone and 4% copper sulphate. Inclusion was achieved with a mixture containing resin EPON Resin 812 polybed F araldite (1:1) and polymerization in an oven at 100° C. for 1 hour. Blocks were cut with an ultra microtome to a thickness of 60-70 nm and copper screens placed on 200 mesh coated with Parlódio film. The grids were counterstained with uranyl acetate and 5% lead citrate and examined in a transmission electron microscope.

MPs were classified according to their diameters and electron dense (ED) or electron lucent (EL) aspect, and semi quantified (0 to 3+) by photo of 30000× magnification.

### Results

Evaluation of MPs in the Endomyocardial Biopsies from Chagasic Patients with Indeterminate Form (IF) Versus with Chronic Cardiac (CC) Disease and Normal Hearts (NH).

The mean numbers of electron dense (ED) MPs and electron lucent (EL) MPs/μm², colloidal gold stained particles (pts.) of archaeal DNA intra and extra MPs/μm² and mean diameter (μm) were quantified in the two group of endomyocardial biopsies and are shown in Table VI:

TABLE VI
Mean numbers of electron dense (ED) MPs and electron lucent
(EL) MPs/μm², colloidal gold stained particles of archaeal DNA
intra and extra MPs/μm² and mean diameter (μm) were
quantified in the two group of endomyocardial biopsies (Student
T test, level of significance P < 0.05)

| mean no./um² | IF | CC | TT (P) |
|---|---|---|---|
| no. MPs/um² | 0.22 (0.21) | 0.11 (0.15) | 0.0013 |
| no. pts extra MPS | 11.14 (9.42) | 6.2 (6.7) | 0.003 |
| no. EDMPs | 0.97 (0.15) | 0.015 (0.04) | 0.05 |
| mean pts./EDMPs | 3.55 (3.27) | 0.38 (0.49) | 0.07 |
| no. ELMPs | 0.04 (0.10) | 0.07 (0.10) | 0.10 |
| Mean pts./ELMPs | 4.57 (4.50) | 1.26 (1.45) | 0.030 |
| Mean diameter EDMPs | 0.70 (0.27) | 0.41 (0.23) | 0.0003 |
| Mean diameter ELMPs | 0.58 (0.28) | 0.46 (0.31) | 0.05 |

The mean number of archaeal DNA intraMPs in Normal hearts (NH) group was 0.34+/−1.80/um2 and extra MPs 2.37+/−4.98. The student T test showed no difference with mean numbers of intraMPs of NH from both IF and CC chagasic myocardium and significantly lower mean numbers of extraMPs from both IF (11.14+/−9.42 p<0.00001) and CC (6.2+/−6.7 p=0.0008).

In biopsies of CC group, there were significant lower numbers of MPs/μm² (0.11+/−0.15) than in IF (0.22+/−0.21), associated with lower numbers of archaeal DNA extra MPs site (6.2+/−6.7 vs 11.14+/−9.42). However CC exhibited higher number of EL MPs/um², with lower numbers of intra archaeal DNA. The ED MPs/μm² were in higher numbers in IF than in CC.

There was a significant difference in the size and archaeal DNA content between these two clinical groups. The EDMPs in IF have higher diameter (0.70+/−0.27 vs 0.41+/−0.23, p=0.30) with higher mean number of intra archaeal DNA content (3.55+/−3.27 vs 0.38+/−0.49).

The EL MPs in CC presented lower numbers of archaeal DNA particles than IF (1.26+/−1.45 vs 4.57+/−4.50, p=0.03), and lower mean diameter (0.46+/−0.31 vs 0.58+/−0.28).

A positive correlation was detected between numbers of archaea DNA intra EL versus extra MPs, in IF (R=0.66), and more strongly correlated in CC(R=0.89). Differently, a positive correlation archaea DNA intra ED MPs and extracellular archaea DNA was seen in IF (R=0.45), but a negative correlation in CC (R=−0.22). See Table VII, below.

TABLE VII
Correlation between microparticles and archaela
DNA detected in IF and CC Chagas patients

| correlation | IF | CC |
|---|---|---|
| archaeal DNA × DNA pts. intra | R = 0.58 | R = 0.49 |
| to MPs | | |
| number ELMPs × number DNA | R = 0.29 | R = −0.14 |
| pts. extra to MPs | | |
| number DNA pts. intra to | R = 0.45 | R = −0.22 |
| EDMPs × number DNA pts. | | |
| extra to MPs | | |
| number pts. intra to ELMPs × | R = 0.66 | R = 0.89 |
| extra MP pts. | | |
| no. pts intra × extra MPs | R = 0.31 | R = 0.77 |
| no. pts arq cl × no. MPEL | R = 0.82 | R = −0.07 |

Evaluation of MPs Present in the Serum from Indeterminate Form (IF) Versus with Chronic Cardiac (CC) Chagasic Patients.

Semi-quantification of CC vs IF MPs according to diameter are shown in Table VIII.

CC group serum presented higher levels of EL MPs from 101-200 nm and >200 nm in supernatant and ED MPs >200 nm in the pellet. In IF group serum, the pellet and supernatant showed higher numbers of 40-100 nm ED MPs, surrounded by monolayer lipidic membrane, which are possibly archaeal proteassomes.

TABLE VIII
Semi-quantification of CC vs IF MPs according
to diameter of EL MPs and ED MPs

| | EL MPs | | | | ED MPs | | | |
|---|---|---|---|---|---|---|---|---|
| | 101-200 nm | | >200 nm | | 40-100 nm | | >200 nm | |
| Groups | pellet | supernant | pellet | supernant | pellet | supernant | pellet | supernant |
| iF | 0.18 | 0.07 | 0.17 | 0.03 | 1.21 | 1.94 | 0.28 | 0.13 |
| CC | 0.23 | 0.27 | 0.44 | 0.34 | 0.52 | 0.74 | 0.64 | 0.12 |
| TT | 0.62 | 0.06 | 0.07 | 0.04 | 0.10 | 0.05 | 0.01 | 0.95 |

Mean numbers of Mps/photo of IF and in CC chagasic pts were compared by student T test (TT). The CC group of serum presented higher levels of EL MPs from 101-200 nm and >200 nm in supernant and ED MPs > 200 nm in the pellet. In IF group of serum, the pellet showed higher numbers of 40-100 nm ED MPs.

### Discussion

An intriguing aspect in Chagas' disease is why around 30% of the T. cruzi infected individuals present a worse outcome, with development of heart failure and sudden death due to a chronic dilated cardiopathy (CC). It is known that such cardiopathy is related with an exacerbated immune response against T. cruzi antigens, leading to an apparently autoimmune lymphocytic myocarditis. The present study support that MPs containing archaeal DNA might also be related to such an outcome.

Two types of MPs are detectable in the myocardium of chagasic patients, one with electron dense lipidic content (ED) more frequently associated with IF, and electron lucent content (EL) in CC group. The present study determined whether archaeal DNA was also present in these two clinical groups of chagasic myocardium using in situ hybridization.

The in situ hybridization electron microscopy showed MPs surrounded by a double membrane containing an archaeal DNA monolayer lipidic membrane nanovesicle. These MPs are present in the myocardium from both asymptomatic indeterminate form (IF) and heart failure CC form of the disease. The archaeal nanovesicles were also observed external to the MPs (i.e., extra MPs). It was possible to demonstrate significant differences between CC versus IF clinical forms regarding the ultra structural characteristic of the MPs. The amount of archaeal DNA was significantly lower in CC than IF group, and the EL MPs archaeal DNA correlated negatively with the amount of archaeal DNA in the extra MPs matrix. These findings strongly support that chagasic patients present different MPs in the myocardium dependent on the clinical form, differences related with archaeal DNA and ultra structural morphologies.

The ED MPs may be related to the positive outcome of IF chagasic patients, and have a similar morphology of organelles described in Trypanosoma cruzi as lysosome-related organelles, which contain many enzymes, including carboxipeptidases^{xxvi}. Presence of family M32 metallocarboxipeptidases (MCP), which is known to be specific to the Archaea and Bacteria kingdom, was described in Trypanosoma cruzi²². The authors suggest that the protozoan has acquired by horizontal transfer of an ancestral gene from an archaea, as the T. cruzi MCP has high biochemical similarity with Archaeon P. furiosus (Pfu) one. Pfu M32 MCP seems to be involved in the utilization of peptides and proteins for nutrition of the organism^{xxvii}. Additionally, studies of T. cruzi genome found sequences of both types of proteasomos, 20S proteasome and hsIV, suggesting the presence of endosymbionts, for example, an α-proteobacterial progenitor of mitochondria or horizontal gene transfer by temporal association with bacteria, within the intestines of insects^{xxviii}. The life cycle of T. cruzi in the myocardial fibers suffers profound morphological changes, using proteins from the host cell. The essential role of proteasomos in the activity for degrading the cytoplasmic proteins has been demonstrated^{xxix},^{xxx}. It is interesting that, in spite of apparently normal hearts, the IF myocardium presents significantly higher amount of extra MPs archaeal DNA nanovesicles, which means that this abnormal values of archaeal nanovesicles may be protective against the presence of abnormal proteins.

The EL MPs, present mainly in CC patients, were related with archaea DNA in nanovesicles among collagen fibers. The possibility of these nanovesicles are carrying archaeal collagenases might explain fragmentation of the collagen and myocardial dilatation. Large EL MPs forms were seen in CC group¹⁸ amidst the chronic inflammatory infiltrate, sometimes with a periplasmic space containing nanoarchaeal-like bodies, in a similar morphology of Ignicoccus hospitalis^{xxxi}. In previous work Chlamydophila pneumonia (Cp) was observed in chagasic myocarditis. Such inflammatory infiltrate is constituted mainly by CD8+ T cells in the presence of T. cruzi antigens, with lack of CD4+ T cells^{xxxii}, ^{xxxiii}. T. cruzi and/or Cp antigens entrapped by liposomes from these archaea (called archaeosomes) may be inducing exacerbated inflammatory infiltrate. It is known that archaeosomes are superior adjuvants that induce long-term CD8+ cytotoxic T cell response to entrapped soluble protein in the absence of CD4+ T cell help^{xxxiv}.

In the present study, analysis of sera showed a higher amount of small ED microparticles (from 40-100 nm, compatible with exosomes) in IF group. EL MPs were seen in greater amount in CC serum group, having from 100 nm to 1 um diameter. A preliminary study demonstrated high levels of archaeal collagenase AMZ1 in the supernatant of CC group.

MPs releasing archaea collagenases may be acting in other disorders such as in rupturing atheroma plaques. The EL MP morphology described in the present study in chagasic cardiopathy is similar to those archaeal-like bodies observed in vulnerable atheroma plaques^{xxxv}, in association with myxoid matrix. These archaea are associated with matrix metallopeptidase (MMP9) and possibly archaeal collagenases.

Summarizing, ultrastructural study of double surrounding membrane

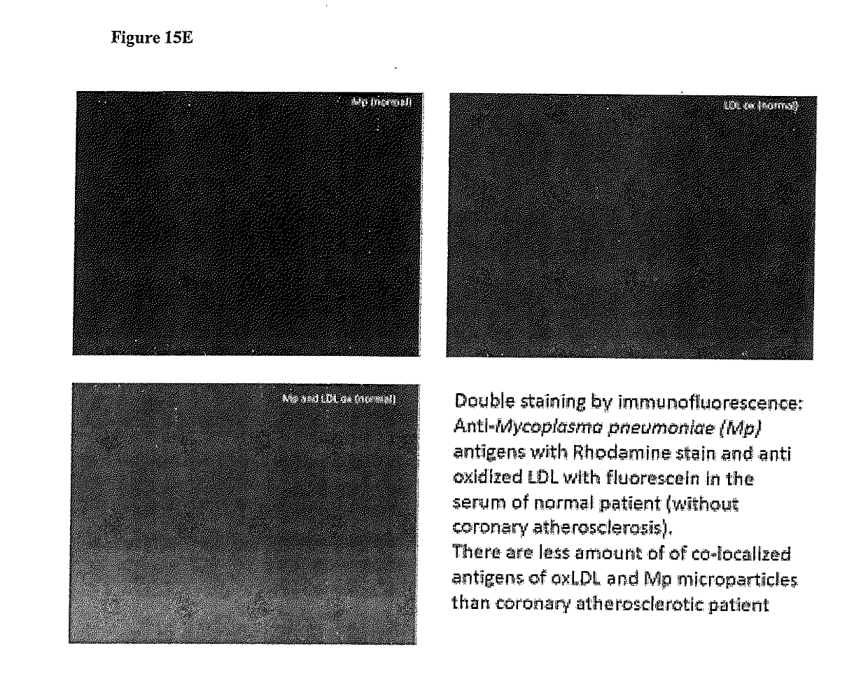

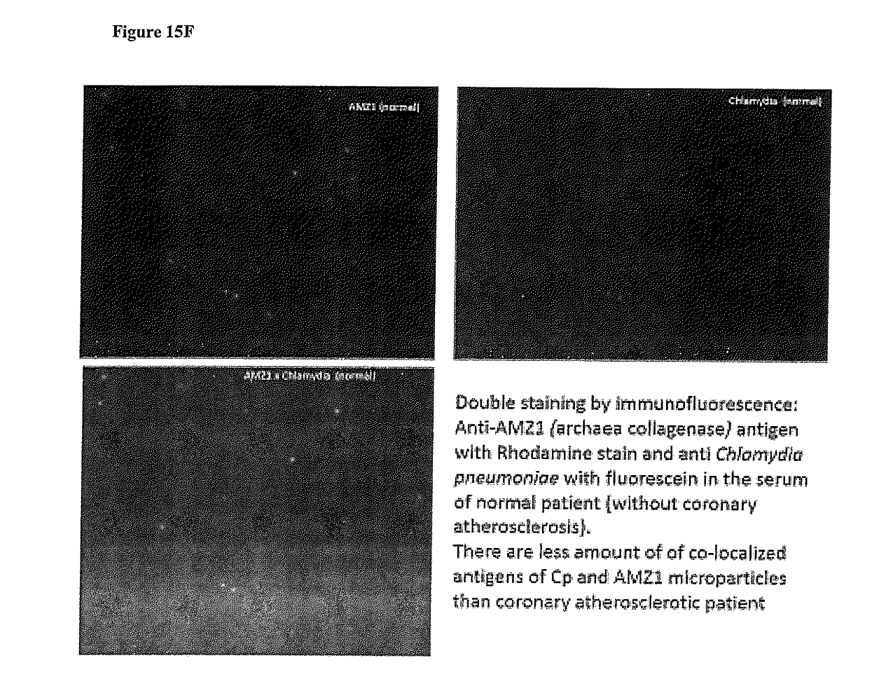

Microparticles suggest that:
a. Pathogenic archaea related with development of heart failure in CC group have clear electron lucent content and lower diameter that those seen in IF with lower intra archaea DNA, and are strongly associated with extra archaea DNA nanovesicles (possibly archaeal collagenases such as AMZ1) that fragment the collagen.
b. Protective archaeal MPs related with asymptomatic IF patients present electron dense content and higher diameter than those seen in CC, richer archaeal DNA content, possibly carrying proteasomes that rid abnormal proteins such as Cp (see FIG. 15).

### Conclusions

Ultra structure was able to differentiate myocardial tissue and serum MPs from chagasic patients with CC and IF states, and both from normal donor hearts. The study revealed that MPs may be differentiated by the morphology, electron dense lipidic content (ED MPs) versus clear empty electron lucent content (EL MPs), double envoltory membrane and archaeal DNA, favouring the hypothesis that these MPs are archaea microbes. Both chagasic groups have higher amount of extra MPs archaeal DNA compared with normal donor hearts. CC patients have higher levels of EL MPs with lower archaeal DNA content, but strongly correlated with the levels of extra MPs archaeal DNA. The present study indicates that these archaea contain archaeal metaloprotease AMZ1. Analysis of MPs from heart or serum samples can be analyzed to characterize patients who will develop worse outcome (i.e. CC) in T. cruzi infected patients. Additionally, removal of EL MPs may be a therapeutic target.

### REFERENCES FOR EXAMPLE 12

¹Colombo E, Borgiani B, Verderio C, Furlan R. Microvesicles: novel biomarkers for neurological disorders. Front Physiol. 2012: 3:63.
²Mastronardi M L, Mostefai H A, Meziani F, Martinez M C, Asfar P, Andriantsitohaina R. Circulating microparticles from septic shock patients exert differential tissue expression of enzymes related to inflammation and oxidative stress. Crit. Care Med. 2011: 39(7):1739-48.
³Bulut D, Scheeler M, Niedballa L M, Miebach T, Mügge A. Effects of immunoadsorption on endothelial function, circulating endothelial progenitor cells and circulating microparticles in patients with inflammatory dilated cardiomyopathy. Clin Res Cardiol. 2011: 100(7):603-10.
⁴Sinning J M, Losch J, Walenta K, Böhm M, Nickenig G, Werner N. Circulating CD31+/Annexin V+ microparticles correlate with cardiovascular outcomes. Eur Heart J. 2011: 32(16):2034-41.
⁵Orozco A F, Lewis D E. Flow cytometric analysis of circulating microparticles in plasma. 2010; Cytometry A. 77(6): 502-14.
⁶Pereira Barrett A C, Mady C, Arteaga-Fernandez E, Stolf N, Lopes E A, Higuchi M L, Bellotti G, Pileggi F. Right ventricular endomyocardial biopsy in chronic Chagas' disease. Am Heart J. 1986: 111(2):307-12.
⁷Higuchi M L, De Morais C F, Pereira Barret A C, Lopes E A, Stolf N, Bellotti G, Pileggi F. The role of active myocarditis in the development of heart failure in chronic Chagas' disease: a study based on endomyocardial biopsies. Clin Cardiol. 1987: 10(10:665-70.
⁸Higuchi M L, Lopes E A, Pereira-Barreto A C, Stolf N, Bellotti G, Verginelli G, Pileggi F. Biópsia endomiocárdica do ventriculo direito: Significado das alterações ultra-estruturais na cardiopatia chagásica. Arq Bras Cardiol 1985: 44:171-175
⁹Higuchi M de L, Gutierrez P S, Aiello V D, Palomino S, Bocchi E, Kalil J, Bellotti G, Pileggi F., Immunohistochemical characterization of infiltrating cells in human chronic chagasic myocarditis: Comparison with myocardial rejection process. Virchows Arch A Pathol Anat Histopathol. 1993: 423:157-60.
¹⁰Higucht M L; Britto T; Reis M M, Barbosa A., Bellotti G, Pereira Barret A C, Pileggi F. Correlation between Trypanosoma cruzi parasitism and myocardial inflammatory infiltrate in human chronic chagasic myocarditis: Light microscopy and immunohistochemical findings. Cardiovasc Pathol 1993: 2:101-106.
¹¹Bellotti G, Bocchi E A, de Moraes A V, Higuchi M L, Barbero-Marcial M, Sosa E, Esteves-Filho A, Kalil R, Weiss R, Jatene A, Pileggi F. In vivo detection of Trypanosoma cruzi antigens in hearts of patients with chronic Chagas' heart disease. Am Heart. J. 1996: 131(2):301-7.
¹²Jones E M, Colley D G, Tostes S, Lopes E R, Vnencak-Jones C L, McCurley T L. Amplification of a Trypanosoma cruzi DNA sequence from inflammatory lesions in human chagasic cardiomyopathy. Am J Trop Med. Hyg. 1993; 48(3):348-57.
¹³Gao W, Luquetti A O, Pereira M A. Immunological tolerance and its breakdown in Chagas' heart disease: role of parasitokines. Front Biosci. 2003: 8: e218-27.
¹⁴Cunha-Neto E, Duranti M, Gruber A, Zingales B, De Messias I, Stolf N, Bellotti G, Patarroyo M E, Pilleggi F, Kalil J. Autoimmunity in Chagas disease cardiopathy: biological relevance of a cardiac myosin-specific epitope crossreactive to an immunodominant Trypanosoma cruzi antigen. Proc Natl Acad Sci USA. 1995; 92(8):3541-5.
¹⁵Higuchi M L, Benvenuti L A, Reis M M, Metzger M. Pathophysiology of the heart in Chagas'disease: current status and new developments. Cardiovasc Res 2003: 60:96-107.
¹⁶Marin-Neto J A, Cunha-Neto E, Maciel B C, Simoes M V. Pathogenesis of chronic Chagas heart disease. Circulation. 2007: 115(9):1109-23.
¹⁷Higuchi M L, Reis M M, Aiello V D, Benvenutti L A Gutierrez P S, Bellotti G, Pileggi F Asssociation of an increase in CD8+ T cells with the presence of Trypanosoma cruzi antigens in chronic, human, chagasic myocarditis. Am J Trop Med Hyg 1997: 56:485-89.
¹⁸Madigan, M. T., J. M. Martinko, and J. Parker. 2000. Prokaryotic diversity: the Archaea In M. T. Madigan, J. M. Martinko, and J. Parker (ed.), Brock biology of microorganisms. Prentice-Hall, Inc., Upper Saddle River, N.J. 33), p. 546-572.
¹⁹DeLong E F. Archaea in coastal marine environments. Proc Natl Acad Sci USA. 1992: 89(12):5685-9.
²⁰Srmivasan V, Morowitz H J. Ancient genes in contemporary persistent microbial pathogens. Biol Bull. 2006: 210:1-9.
²¹Niemirowicz G, Parussini F, Aguero F, Cazzulo J J. Two metallocarboxypeptidases from the protozoan Trypanosoma cruzi belong to the M32 family, found so far only in prokaryotes. Biochem J. 2007: 401(2):399-410.
²²Gille C, Goede A, Schloetelburg C, Preissner R, Kloetzel P M, Gabel U B, Frömmel C. A comprehensive view on proteasomal sequences: implications for the evolution of the proteasome. J Mol Biol. 2003: 326(5):1437-48.
²³Yamada S, Niwa J, Ishigaki S, Takahashi M, Ito T, Sone J, Doyu M, Sobue G. Archaeal proteasomos effectively degrade aggregation-prone proteins and reduce cellular toxicities in mammalian cells, J Biol Chem 2006: 281, 23842-23851.
²⁴Bustamante E, Pediaditakis P, He L, Lemasters J J. Isolated mouse liver mitochondria are devoid of glucokinase. Biochem Biophys Res Commun. 2005: 334(3):907-10.
²⁵Duarte M I S, Mariano O N, Micalaza C F H, Uip D E, Corbett C E P. A fast method for processing biologic material for electron microscopic diagnosis in infectious disease. Ultrastruct Pathol, 1992; 16: 475-482.
²⁶SantlAnna C, Parussini F, Lourenco D, de Souza W, Cazzulo J J, Cunha-e-Silva N L. All Trypanosoma cruzi developmental forms present lysosome-related organelles. Histochem Cell Biol. 2008: 130(6):1187-98.
²⁷Schul G, Brehm S D, Dallas S, Adams M W Whole-genome DNA microarray analysis of a hyperthermophile and na archaeon: Pyrococcus fusiosus grown on carbohidrates or peptides. J Bacteriol 2003: 185:3935-47.
²⁸Gille C, Goede A, Schlöetelburg C, Preissner R, Kloetzel P M, Gabel U B, Frommel C. A comprehensive view on proteasomal sequences: implications for the evolution of the proteasome. J Mol Biol. 2003: 326(5):1437-48.
²⁹González J, Ramalho-Pinto F J, Frevert U, Ghiso J, Tomlinson S, Scharfstein J, Corey E J, Nussenzweig V. Proteasome activity is required for the stage-specific transformation of a protozoan parasite. J Exp Med. 1996: 184(5):1909-18.
³⁰De Diego J L, Katz J M, Marshall P, Bessy G, Manning J, Nussenzweig V, Gonzalez J. The ubiquitina-proteasome pathway plays an essential role in proteolysis during Trypanosoma cruzi remodeling. Biochem 2001: 40:1053-1062.
³¹Junglas B, Briegel A, Burghardt T, Walther P, Wirth R, Huber H, Rachel R. Ignicoccus hospitalis and Nanoarchaeum equitans: ultrastructure, cell-cell interaction, and 3D reconstruction from serial sections of freeze-substituted cells and by electron cryotomography. Arch Microbiol. 2008: 190(3):395-408.
³²Higuchi M L, Reis M M, Aiello V D, Benvenutti L A Gutierrez P S, Bellotti G, Pileggi F Asssociation of an increase in CD8+ T cells with the presence of Trypanosoma cruzi antigens in chronic, human, chagasic myocarditis. Am J Trop Med Hyg. 1997: 56:485-89.
³³Reis M M, Higuchi Mde L, Benvenuti L A, Aiello V D, Gutierrez P S, Bellotti G, Pileggi F. An in situ quantitative immunohistochemical study of cytokines and IL-2R+ in chronic human chagasic myocarditis: correlation with the presence of myocardial Trypanosoma cruzi antigens. Clin Immunol Immunopathol. 1997: 83(2):165-72.
³⁴Krishnan L, Sad S, Patel G B, Sprott G D. Archaeosomes induce long-term CD8+ cytotoxic T cell response to entrapped soluble protein by the exogenous cytosolic pathway, in the absence of CD4+ T cell help. J. Immunol. 2000: 165(9):5177-85.
³⁵Higuchi M L, Santos M H, Roggerio A, Kawakami J T, Bezerra H G, Canzian M. A role for archaeal organisms in development of atherosclerotic vulnerable plaques and myxoid matrices. Clinics (Sao Paulo). 2006: 61(5):473-8.

### Example 13

### Co-Localization of Mycoplasma Pneumoniae and Oxidized LDL or Chlamydia Pneumoniae and Archaea Collagenase (AMZ1) in Serum Samples from Subjects with Vulnerable Atherosclerotic Plaque

Serum samples were collected from subjects diagnosed with vulnerable atherosclerotic plaque or stable atherosclerotic plaque, as well as from healthy subjects. Serum samples were analyzed using immunohistochemistry to detect Mycoplasma pneumoniae and oxidized LDL, or to detect Chlamydia pneumoniae and archaea collagenase (AMZ1) in the subject samples. As shown in FIG. 15, serum samples from subjects diagnosed with vulnerable atherosclerotic plaque exhibited greater levels of co-localization of Mycoplasma pneumoniae and oxidized LDL in microparticles in the serum samples, or greater levels of co-localization of Chlamydia pneumoniae and archaea collagenase in microparticles in the serum samples, compared to subjects diagnosed with stable atherosclerotic plaque or who were healthy. The results are also summarized in Table VIII below.

TABLE VIII
Semi-quantification of CC vs IF MPs according
to diameter of EL MPs and ED MPs

| | Amount of Microparticles in the serum | |
|---|---|---|
| | detected by double immunofluorescence | |
| | Co-localized antigens | Co-localized antigens |
| | from Micoplasma | from AMZ1/Chlamydia |
| Subject | pneumoniae/LDL OX | pneumoniae |
| Atherosclerotic with | Absent/Low level | Medium level |
| stable plaque | | |
| Atherosclerotic with | High level | High level |
| vulnerable plaque | | |
| (great risk | | |
| of Infarction) | | |

### Example 14

### Heart Failure in Chagas Disease Patients is Associated with Microparticles, Archaeal DNA and Archaeal Collagenase in the Serum

### Method

Two groups of sera from chagasic pts, indeterminate form (IF) (n=8) and heart failure (HF) (n=7), were centrifuged obtaining a pellet with dense MPs, which was processed for electron microscopy (EM) and immunoelectronic anti-archaemetzincin-1 (AMZ1, Novas Biologicals) and ARCH 915 probe in situ hybridization techniques, linked with 10 nm colloidal gold. The mean positive dots/photo of 50,000× magnification/case was obtained.

### Results

The mean number of MPs in HF versus IF groups did not differ, however there was a higher number of AMZ1 immunogold positive dots outside MPs, in the pellet of HF than IF group (table IX). The mean numbers of MPs exhibited a positive correlation with numbers of archaeal DNA outside MPs in HF sera but not in IF.

### Conclusion

HF chagasic patients group of sera could be differentiate by the increased amount of collagenase AMZ1 external to the microparticles, detected at electron microscopy, tha in IF. MPs in HF group seem to correspond to pathogenic archaea, releasing their DNA and collagenase to the extracellular medium. Further studies may reveal if the increased amount of archaeal AMZ1 in the serum may be biomarkers of HF.

TABLE IX
Mean number/EM photo of Microparticles (MP), AMZ1 positive
dots extra MPs, and Archaeal DNA positive dots (Arch DNA)
extra MPs, in the pellet of sera from Heart Failure (HF)
and Indeterminate Form (IF) of chagasic patients.

| | HF | IF | p= |
|---|---|---|---|
| MPs | 7.5 | 7.3 | 0.48 |
| AMZ1 | 79.2 | 15.2 | 0.01 |
| Arch DNA | 15.4 | 11.3 | 0.35 |
| correl no. | 0.95 (0.001) | −0.370 (0.76) | |
| MPs × Arch | | | |
| DNA − R (p) | | | |

### Example 15

### Archaeosomal Microparticles Comprising AMZ1 Present in Heart Failure in Chagasic Patients with Asymptomatic Indeterminate Form

Microparticles (MPs) in the serum have been related with the presence of Heart Failure (HF). HF occurs in 30% of Trypanosoma cruzi infected chagasic individuals. As described in Example 12, we found in chagasic endomyocardial biopsies that archaeal gene encoded electron dense lipidic (ED) organelles are possibly archaeosomes that entrap extracellular proteins having the function of ridding abnormal proteins, and are increased in asymptomatic indeterminate form (IF) patients. Electron lucent pathogenic archaea were increased in HF patients, possibly releasing metalloprotease. Here we searched if archaeosomes are increased in the serum of IF, entrapping Archaemetzincin-1 (AMZ1), a metalloprotease widely seen in arehaea, compared with serum of heart failure (HF).

### Material and Methods

Sera from 8 HF and 7 from IF chagasic patients were submitted to a technique of MP separation, in a mannitol/sucrose rich solution. After centrifugation, MPs in the pellet and in the supernatant were studied at immunoeletron microscopy, using anti-AMZ1 monoclonal antibody (Novas Biologicals). The mean number/μm² of ED MPs <100 nm and of AMZ1 positive dots intra or extra ED MPs were obtained from 10 photos/case in 50K magnification.

### Results

In the supernatant, ED MPs were present in higher numbers in IF (33.4±50.9) than in HF (0.2±1.1), P<0.001; in IF the ED MPs contained AMZ1 positive dots (1.6±3.7), in positive correlation with numbers of ED MPs (r=0.47, P<0.0001) and in HF, ED MPs were almost absent and did not contain AMZ1 dots. In the pellet, the amount of ED MPs did not differ between HF versus IF groups (3.6±5.9 vs 2.5±4.9, P=0.74), but AMZ1 positive dots extra ED MPs were significantly increased in HF (80.5±132.3) compared to IF (15.5±19.27), P<0.001. Also, in the pellet, numbers of ED MPs correlated negatively with AMZ1 extra ED MPs in HF (r=−0.63, P<0.001), but not in IF (r=0.13, P=0.34).

### Conclusion

ED MPs <100 nm in the serum of IF chagasic patients seem to be archaeosomes, which remove free metalloprotease particles from the serum, preventing HF whereas the absence of the ED MPs is associated with increase of free metalloprotease in serum of HF patients. This is a first human documentation of removal of free abnormal protein from the serum by archaeosomes.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

Patents, patent applications, publications, product descriptions and protocols that may be cited throughout this application, the disclosures of which are incorporated herein by reference in their entireties for all purposes.

What is claimed is:

1. A method of diagnosing heart disease or heart failure in a subject comprising detecting the presence of electron dense microparticles comprising archael collagenase in a sample from the subject, wherein the presence of the electron dense microparticles comprising archael collagenase indicates diagnosis of heart disease or heart failure.

2. The method of claim 1, wherein the electron dense microparticles are archaeosomes.

3. A method for diagnosing heart disease or heart failure in a first subject comprising:
(a) obtaining a first sample from the first subject;
(b) obtaining a second sample from a second subject, wherein the second subject does not have heart disease or heart failure, and wherein the second sample comprises microparticles;
(c) detecting the presence of microparticles in the first and second samples,
wherein the presence of a fewer number of electron dense microparticles comprising archael collagenase in the first sample compared to the second sample indicates a diagnosis of heart disease or heart failure in the first subject.

4. A method for diagnosing heart disease or heart failure in a subject comprising:
(a) obtaining a sample from the first subject;
(b) determining the number of microparticles in the sample,
(c) comparing the number of electron dense microparticles comprising archael collagenase in the sample to a reference value determined using one or more subject that does not have heart disease or heart failure;
wherein the presence of a fewer number of electron dense microparticles comprising archael collagenase in the sample compared to the reference value indicates a diagnosis of heart disease or heart failure in the subject.

5. The method of claim 3, wherein the heart disease or heart failure is associated with Chagas disease.

6. The method of claim 3, wherein the first and second samples are each selected from the group consisting of serum, blood, plasma and an endomyocardial sample.

7. The method of claim 3, wherein the archaeal collagenase is Archeobacterial metalloproteinase-like protein 1 (AMZ1).

8. The method of claim 3, wherein the second subject has the indeterminate form of Chagas disease.

9. A method of diagnosing atherosclerosis with vulnerable plaque in a subject comprising detecting the co-localization of two or more agents in a sample from the subject, wherein each agent is independently selected from the group consisting of Mycoplasma pneumoniae, oxidized low-density lipoprotein, Chlamydia pneumoniae, and archaea collagenase, wherein presence of the co-localized agents indicates diagnosis of atherosclerosis with vulnerable plaque.

10. The method of claim 9, wherein the co-localized agents comprise Mycoplasma pneumoniae and oxidized low-density lipoprotein.

11. The method of claim 9, wherein the co-localized agents comprise Chlamydia pneumoniae and archaea collagenase.

12. The method of claim 9, wherein the archaeal collagenase is Archeobacterial metalloproteinase-like protein 1 (AMZ1).

13. A method for diagnosing atherosclerosis with vulnerable plaque in a first subject comprising:
(a) obtaining a first sample from the first subject;
(b) obtaining a second sample from a second subject who does not have atherosclerosis with vulnerable plaque;
(c) determining the number of co-localized agents in the first and second samples, wherein each agent is independently selected from the group consisting of Mycoplasma pneumoniae, oxidized low-density lipoprotein, Chlamydia pneumoniae, and archaea collagenase, and
wherein the presence of a greater number of co-localized agents in the first sample compared to the second sample indicates a diagnosis of atherosclerosis with vulnerable plaque in the first subject.

14. A method for diagnosing atherosclerosis with vulnerable plaque in a subject comprising:
(a) obtaining a sample from the subject;
(b) determining the number of co-localized agents in the sample, wherein each agent is independently selected from the group consisting of Mycoplasma pneumoniae, oxidized low-density lipoprotein, Chlamydia pneumoniae, and archaea collagenase; and
(c) comparing the number of co-localized agents in the sample with a reference value determined using one or more subject who does not have atherosclerosis with vulnerable plaque;
wherein the presence of a greater number of co-localized agents in the sample compared to the reference value indicates a diagnosis of atherosclerosis with vulnerable plaque in the subject.

15. A method for diagnosing heart disease or heart failure in a first subject comprising:
(a) obtaining a first sample from the first subject;
(b) obtaining a second sample from a second subject, wherein the second subject does not have heart disease or heart failure;
(c) detecting the presence of microparticles in the first and second samples,
wherein the presence of a greater number of electron lucent microparticles associated with archaeal collagenase in the first sample compared to the second sample indicates a diagnosis of heart disease or heart failure in the first subject.

16. A method for diagnosing heart disease or heart failure in a subject comprising:
(a) obtaining a sample from the subject;
(b) determining the number of electron lucent microparticles associated with archael collagenase in the sample; and
(c) comparing the number of electron lucent microparticles associated with archael collagenase in the sample with a reference value determined using one or more subject that does not have heart disease or heart failure;
wherein the presence of a greater number of electron lucent microparticles associated with archaeal collagenase in the sample compared to the reference value indicates a diagnosis of heart disease or heart failure in the subject.

17. The method of claim 11, wherein the archaeal collagenase is archaelysin family metallopeptidase 1 (AMZ1).

18. The method of claim 11, wherein the first subject has Chagas disease.

19. The method of claim 10 wherein the first subject is in treatment for malignant neoplasia.

20. The method of claim 11 wherein the first subject is in treatment for malignant neoplasia.


SEQUENCE LISTING (5 sequences)

SEQ ID NO: 1 - DNA, 28 bp, Artificial Sequence
  Synthetic oligonucleotide
ggaattccatatggcacccggatcgagc

SEQ ID NO: 2 - DNA, 34 bp, Artificial Sequence
  Synthetic oligonucleotide
cggatccgggcgtacttctttcactggtgccggt

SEQ ID NO: 3 - DNA, 2010 bp, Artificial Sequence
  Variant of T. Cruzi trans-sialidase gene.
atgggcagcagccatcatcatcatcatcacagcagcggcctggtgccgcgcggcagccatatggcacccggatcgagccgagttgagctgtttaagcggcaaagctcgaaggtgccatttgaaaagggcggcaaagtcaccgagcgggttgtccactcgttccgcctccccgcccttgttaatgtggacggggtgatggttgccatcgcggacgctcgctacgaaacatccaatgacaactccctcattgatacggtggcgaagtacagcgtggacgatggggagacgtgggagacccaaattgccatcaagaacagtcgtgcatcgtctgtttctcgtgtggtggatcccacagtgattgtgaagggcaacaagctttacgtcctggttggaagctacaacagttcgaggagctactggacgtcgcatggtgatgcgagagactgggatattctgcttgccgttggtgaggtcacgaagtccactgcgggcggcaagataactgcgagtatcaaatgggggagccccgtgtcactgaaggaatttttcccggcggaaatggaaggaatgcacacaaatcaatttcttggcggtgcaggtgttgccattgtggcgtccaacgggaatcttgtgtaccctgtgcaggttacgaacaaaaagaagcaagttttttccaagatcttctactcggaagacgagggcaagacgtggaagtttggggagggtaggagtgattttggctgctctgaacctgtggcccttgagtgggaggggaagctcatcataaacactcgagttgactatcgccgccgtctggtgtacgagtccagtgacatggggaattcgtgggtggaggctgtcggcacgctctcacgtgtgtggggcccctcaccaaaatcgaaccagcccggcagtcagagcagcttcactgccgtgaccatcgagggaatgcgtgttatgctcttcacacacccgctgaattttaagggaaggtggctgcgcgaccgactgaacctctggctgacggataaccagcgcatttataacgttgggcaagtatccattggtgatgaaaattccgcctacagctccgtcctgtacaaggatgataagctgtactgtttgcatgagatcaacagtaacgaggtgtacagccttgtttttgcgcgcctggttggcgagctacggatcattaaatcagtgctgcagtcctggaagaattgggacagccacctgtccagcatttgcacccctgctgatccagccgcttcgtcgtcagagcgtggttgtggtcccgctgtcaccacggttggtcttgttggctttttgtcgcacagtgccaccaaaaccgaatgggaggatgcgtaccgctgcgtcaacgcaagcacggcaaatgcggagagggttccgaacggtttgaagtttgcgggggttggcggaggggcgctttggccggtgagccagcaggggcagaatcaacggtatcactttgcaaaccacgcgttcacgctggtggcgtcggtgacgattcacgaggttccgagcgtcgcgagtcctttgctgggtgcgagcctggactcttctggtggcaaaaaactcctggggctctcgtacgacgagaagcaccagtggcagccaatatacggatcaacgccggtgacgccgaccggatcgtgggagatgggtaagaggtaccacgtggttcttacgatggcgaataaaattggttcggtgtacattgatggagaacctctggagggttcagggcagaccgttgtgccagacgggaggacgcctgacatctcccacttctacgttggcgggtatggaaggagtgatatgccaaccataagccacgtgacggtgaataatgttcttctttacaaccgtcagctgaatgccgaggagatcaggaccttgttcttgagccaggacctgattggcacggaagcacacatgggcagcagcagcggcagcagtgaaagaagtacgcccggatccggctgctaa

SEQ ID NO: 4 - protein, 669 aa, Artificial Sequence
  Variant of T. Cruzi trans-sialidase protein.
MGSSHHHHHHSSGLVPRGSHMAPGSSRVELFKRQSSKVPFEKGGKVTERVVHSFRLPALVNVDGVMVAIADARYETSNDNSLIDTVAKYSVDDGETWETQIAIKNSRASSVSRVVDPTVIVKGNKLYVLVGSYNSSRSYWTSHGDARDWDILLAVGEVTKSTAGGKITASIKWGSPVSLKEFFPAEMEGMHTNQFLGGAGVAIVASNGNLVYPVQVTNKKKQVFSKIFYSEDEGKTWKFGEGRSDFGCSEPVALEWEGKLIINTRVDYRRRLVYESSDMGNSWVEAVGTLSRVWGPSPKSNQPGSQSSFTAVTIEGMRVMLFTHPLNFKGRWLRDRLNLWLTDNQRIYNVGQVSIGDENSAYSSVLYKDDKLYCLHEINSNEVYSLVFARLVGELRIIKSVLQSWKNWDSHLSSICTPADPAASSSERGCGPAVTTVGLVGFLSHSATKTEWEDAYRCVNASTANAERVPNGLKFAGVGGGALWPVSQQGQNQRYHFANHAFTLVASVTIHEVPSVASPLLGASLDSSGGKKLLGLSYDEKHQWQPIYGSTPVTPTGSWEMGKRYHVVLTMANKIGSVYIDGEPLEGSGQTVVPDGRTPDISHFYVGGYGRSDMPTISHVTVNNVLLYNRQLNAEEIRTLFLSQDLIGTEAHMGSSSGSSERSTPGSGC

SEQ ID NO: 5 - DNA, 20 bp, Artificial sequence
  Synthetic oligonucleotide
gtgctcccccgccaattcct